(19) United States
(12) Patent Application Publication
(10) Pub. No.: US 2009/0143417 A1
(43) Pub. Date: Jun. 4, 2009

(54) METHODS OF TREATING PAIN

(75) Inventors: Maree Therese Smith, Queensland (AU); Craig McKenzie Williams, Queensland (AU)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: The University of Queensland, St. Lucia, Queensland (AU)

(21) Appl. No.: 11/794,137

(22) PCT Filed: Dec. 23, 2005
(86) PCT No.: PCT/AU2005/001976

§ 371 Date: Sep. 2, 2008

(30) Foreign Application Priority Data
Dec. 24, 2004 (AU) 2004907352

(51) Int. Cl. A61K 31/485; A61P 35/00; A61K 31/4468; A61K 31/439; A61K 31/135; C07D 221/28; C07D 489/00

(52) U.S. Cl. 514/282; 514/315; 514/289; 514/646; 546/74; 546/63

(57) ABSTRACT

The present invention is directed to methods and compositions for inducing, promoting or otherwise facilitating pain relief. More particularly the present invention discloses the combination of a nitric oxide donor and an opioid analgesic in the therapeutic management of vertebrate animals including humans, for the prevention or alleviation of pain, particularly moderate to severe pain. In particular, the nitric oxide donor is a slow-release nitric oxide donor or is formulated to provide a sustained release of a low dose of nitric oxide.

### FIELD OF THE INVENTION

This invention relates generally to methods for inducing, promoting or otherwise facilitating pain relief. More particularly the invention relates to the combination of a nitric oxide donor and an opioid analgesic in the therapeutic management of vertebrate animals including humans, for the prevention or alleviation of pain, particularly moderate to severe pain. In particular, the nitric oxide donor is a slow-release nitric oxide donor or is formulated to provide a sustained release of a low dose of nitric oxide.

### BACKGROUND OF THE INVENTION

Opioid analgesics are the most effective class of drugs available for the management of pain. Morphine is the ‘gold standard’ strong opioid analgesic with which all new opioid analgesics are compared. Morphine is also recommended by the World Health Organisation as the drug of choice for the relief of moderate to severe cancer pain, the alleviation of moderate to severe pain in the post-surgical setting and for the relief of pain following trauma and cardiac infarction.

However, the opioid analgesics, including morphine, are well documented to produce a range of unwanted side effects. Severe side effects include allergic reactions, such as difficulty breathing, swelling of lips, tongue, face and/or throat and hives; respiratory depression; seizures; cold, clammy skin; severe weakness, severe dizziness; and unconsciousness. Other side effects include sedation, nausea, vomiting, dry mouth, loss of appetite, constipation, dizziness, tiredness, lightheadedness, muscle twitching, sweating, pruritis, urinary retention and loss of libido. Furthermore, long term use of opioid analgesics can result in tolerance where increasing amounts of opioid analgesics are required to provide a constant level of pain relief. Some opioid analgesics, such as morphine, may upon moderate or long term use, also result in patient dependency.

There is a need for therapies that deliver the pain relief of opioid analgesics with reduced side effects. There is also a need for therapies that reduce opioid analgesic consumption but still provide adequate pain relief.

### SUMMARY OF THE INVENTION

The present invention is predicated in part on the determination that very low biological concentrations of nitric oxide increase the pain relieving potency of opioid analgesics and/or the duration of analgesia achieved by opioid analgesics thereby allowing a given amount of opioid analgesic to achieve longer-lasting pain relief or allowing less opioid analgesic to be used to achieve a given level of pain relief.

Accordingly, in one aspect of the invention there is provided a method of producing analgesia in a subject comprising administering to a subject an effective amount of a nitric oxide donor and an effective amount of an opioid analgesic, wherein the effective amount of nitric oxide donor delivers nitric oxide at a rate of 0.0002 nmol/kg/hour to 2.0 nmol/kg/hour. Preferably the nitric oxide donor is a slow-release nitric oxide donor or is formulated in a sustained release formulation.

In a further aspect of the invention there is provided a method of producing analgesia in a subject comprising administering to a subject an effective amount of a nitric oxide donor formulated in a sustained release formulation and an effective amount of an opioid analgesic, wherein the sustained release formulation of the nitric oxide donor delivers nitric oxide at a rate of 0.0002 nmol/kg/hour to 2.0 nmol/kg/hour.

In yet a further aspect of the present invention there is provided a method of producing analgesia in a subject comprising administering to the subject an effective amount of a slow-release nitric oxide donor and an effective amount of an opioid analgesic wherein the effective amount of slow-release nitric oxide donor is in the range of 0.004 nmol/kg to 0.4 mmol/kg.

In another aspect, the invention provides methods of producing analgesia in a subject comprising administering an effective amount of a slow-release nitric oxide donor or a sustained release formulation of a nitric oxide donor and a sub-analgesic amount of an opioid analgesic.

In another aspect, the present invention provides methods of producing analgesia in a subject comprising administering an effective amount of a slow-release nitric oxide donor or a sustained release formulation of a nitric oxide donor and an effective amount of an opioid analgesic, wherein the nitric oxide donor releases nitric oxide in the form of NO⁺ or NO⁻.

In yet another aspect, the present invention provides methods of producing analgesia comprising administering an effective amount of a slow-release nitric oxide donor or a sustained release formulation of a nitric oxide donor and an effective amount of an opioid analgesic, wherein the nitric oxide donor enhances the endogenous production of nitrosothiols.

In a further aspect, the present invention provides methods of producing analgesia comprising administering an effective amount of a slow-release nitric oxide donor or a sustained release formulation of a nitric oxide donor and an effective amount of an opioid analgesic, wherein the nitric oxide donor reduces the endogenous production of peroxynitrite.

In yet a further aspect, the present invention provides methods of producing analgesia comprising administering an effective amount of a slow-release nitric oxide donor or a sustained release formulation of a nitric oxide donor and an effective amount of an opioid analgesic, wherein the nitric oxide donor causes more endogenous production of nitrosothiols than endogenous production of peroxynitrite.

In a preferred embodiment, the effective amount of the nitric oxide donor is one that increases the ratio of nitrosothiol concentration:peroxynitrite concentration in a biological fluid, such as blood, serum, plasma, lymph, cerebrospinal fluid or brain extracellular fluid, by a factor of at least 1.1, when compared to the ratio of nitrosothiol concentration:peroxynitrite concentration that is observed upon administration of a sustained release formulation of nitroglycerine which delivers 5 mg of nitroglycerine per 24 hours.

The slow-release nitric oxide donor is administered simultaneously, separately or sequentially with the opioid analgesic to achieve analgesia. The sustained release formulation of nitric oxide donor is administered simultaneously and or separately with the opioid analgesic to achieve analgesia. The opioid analgesic may be administered in an analgesic amount or a sub-analgesic amount. The nitric oxide donor and the opioid analgesic are suitably administered in the form of one or more compositions, each comprising a pharmaceutically acceptable carrier and/or diluent. The composition(s) may be administered by injection, by topical application, by intrathecal administration, epidural administration, intracerebroventricular administration, buccal administration, rectal administration, transdermal administration or by the oral route including sustained-release modes of administration, over a period of time and in amounts which are effective for the production of analgesia in a subject.

The slow-release nitric oxide donor is suitably selected from any substance that is converted or degraded or metabolised into, or provides a source of, in vivo nitric oxide over an extended period of time. In one embodiment, the slow-release nitric oxide donor comprises a nitrato group coupled to a carrier compound by a linker.

The opioid analgesic is suitably selected from any opioid compound having analgesic activity. In one embodiment, the opioid analgesic is selected from morphine, methadone, fentanyl, sufentanil, alfentanil, hydromorphone, oxymorphone, oxycodone, codeine, hydrocodeine, hydrocodone, levorphanol, meperidine, heroin, morphine-6-glucuronide, levallorphan, 6-monoacetylmorphine and traniadol.

In a further aspect, the present invention provides methods of relieving pain, comprising administering an effective amount of a nitric oxide donor and an effective amount of an opioid analgesic wherein the nitric oxide donor is a slow-release nitric oxide donor or is formulated in a sustained release formulation which delivers nitric oxide at a rate of 0.0002 nmol/kg/hour to 2.0 mmol/kg/hour. In particular, these methods are suitable for relief of nociceptive pain such as moderate to severe cancer pain, moderate to severe post-surgical pain, moderate to severe pain caused by trauma due to physical injury or cardiac infarction and inflammatory pain states such as arthritis.

In yet a further aspect, the invention provides the use of a nitric oxide donor and an opioid analgesic in the manufacture of a single medicament or separate medicaments for use in a combination therapy, for producing analgesia and/or for relieving moderate to severe pain, wherein the nitric oxide donor is a slow-release nitric oxide donor or is formulated in a sustained release formulation which delivers nitric oxide at a rate of 0.0002 mmol/kg/hour to 2.0 nmol/kg/hour.

### BRIEF DESCRIPTION OF THE DRAWINGS

FIG. 1 is a graphical representation showing the antinociceptive potency of morphine to a noxious stimulus in naïve rats. A single bolus subcutaneous (s.c.) dose of morphine administered at 10 μmol/kg (open triangles) provides near maximal pain relief from a noxious thermal stimulus using the tail flick test, at one hour. An ED₂₀ dose of morphine (2.8 μmol/kg s.c.) (closed triangles) gives only 20% of the maximal pain relief effect. An ED₂₀ dose of morphine (2.8 μmol/kg s.c.) given in combination with a 0.04 nmol/kg s.c. dose of a slow-release nitric oxide donor, compound 2 produces maximal pain relief within one hour (open diamonds) even though the administration of compound 2 alone (0.04 nmol/kg s.c.) does not provide any significant pain relief (closed diamonds).

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 20%, or 10% to a reference quantity, level, value, dimension, size, or amount.

As used herein, the term “alkyl”, used either alone or in compound words, denotes saturated straight chain, branched or cyclic hydrocarbon groups, preferably C₁₋₂₀ alkyl, eg C₁₋₁₀ or C₁₋₆. Examples of straight chain and branched alkyl include, but are not limited to, methyl, ethyl, propyl, isopropyl, butyl, sec-butyl, tert-butyl, n-pentyl and branched isomers thereof, n-hexyl and branched isomers thereof, n-heptyl and branched isomers thereof, n-octyl and branched isomers thereof, n-nonyl and branched isomers thereof, and n-decyl and branched isomers thereof. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. An alkyl group may be further optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or poly-unsaturated alkyl or cycloalkyl groups as previously defined, preferably C₂₋₂₀ alkenyl (eg C₂₋₁₀ or C₂₋₆). Examples of alkenyl include, but are not limited to, vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethnically mono-, di- or poly-unsaturated alkyl or cycloalkyl groups as previously defined. The term preferably refers to C₂₋₂₀ alkynyl. Examples include, but are not limited to, ethynyl, 1-propynyl, 2-propynyl, and butynyl isomers, and pentynyl isomers. An alkynyl group may be further optionally substituted by one or more optional substituents as herein defined.

The term “acyl” denotes a group containing the moiety C═O (and not being a carboxylic acid, ester or amide). Preferred acyl groups include C(O)—R, wherein R is hydrogen or an alkyl, alkenyl, alkynyl, aryl or heterocyclyl residue, preferably a C₁₋₂₀ residue. Examples of acyl include formyl; straight chain or branched alkanoyl such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanol, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; and heterocyclicalkenoyl such as heterocyclicpropenoyl, heterocyclicbutenoyl, heterocyclicpentenoyl and heterocyclichexenoyl.

As used herein, the terms “alkoxy”, “aryloxy”, “alkenyloxy”, “alkynyloxy”, “acyloxy”, and “heterocycyloxy” denote an alkyl, aryl, alkenyl, alkynyl, acyl or heterocyclyl group as herein defined when linked by an oxygen.

As used herein, the terms “alkylthio”, “alkenylthio”, “alkynylthio”, “arylthio”, “acylthio” or “heteocyclylthio” denote an alkyl, alkenyl, alkynyl, aryl, acyl or heterocyclyl group as herein defined when linked by a sulfur atom.

As used herein, the terms “alkylamino”, “alkenylamino”, “dialkylamino”, “alkenylamino”, “arylamino”, “diarylamino”, “acylamino” and “heterocyclylamino” denote one or two alkyl or aryl groups or an alkenyl, alkynyl, acyl or heterocyclyl group as herein defined when linked by an NH or N atom.

As used herein, the term “aryl” denotes a C₆-C₁₄ aromatic hydrocarbon group. Suitable aryl groups include phenyl, biphenyl, naphthyl, tetrahydronaphthyl, anthracenyl, dihydroanthracenyl and phenanthrenyl. Preferred aryl groups include phenyl and naphthyl. An aryl group may be further optionally substituted by one or more optional substituents.

The term “analgesia” is used herein to describe states of reduced pain perception, including absence from pain sensations as well as states of reduced or absent sensitivity to noxious stimuli. Such states of reduced or absent pain perception are induced by the administration of a pain-controlling agent or agents also called “analgesics” and occur without loss of consciousness, as is commonly understood in the art. The term analgesia encompasses the term “antinociception”, which is used in the art as a quantitative measure of analgesia or reduced pain sensitivity in animal models.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “effective amount”, in the context of treating or preventing pain is meant the administration of that amount of active to an individual in need of such treatment or prophylaxis, either in a single dose or as part of a series, that is effective for the prevention of pain, holding pain in check, and/or treating existing pain. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “halo”, is intended to include fluoro, chloro, bromo and iodo substituents.

The term “heterocyclyl” denotes monocyclic, polycyclic or fused, saturated, unsaturated or aromatic hydrocarbon residues, wherein one or more carbon atoms (and where appropriate, hydrogen atoms attached thereto) are replaced by a heteroatom. Suitable heteroatoms include, O, N, S, and Se. Where two or more carbon atoms are replaced, this may be by two or more of the same heteroatom or by different heteroatoms. Suitable examples of heterocyclic groups may include pyrrolidinyl, pyrrolinyl, piperidyl, piperazinyl, morpholino, indolinyl, imidazolidinyl, pyrazolidinyl, thiomorpholino, dioxanyl, tetrahydrofuranyl, tetrahydropyranyl, tetrahydropyrrolyl, pyridyl, thienyl, furyl, pyrrolyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. A heterocyclyl group may be further optionally substituted by one or more optional substituents as herein defined.

“Nociceptive pain” refers to the normal, acute pain sensation evoked by activation of nociceptors located in non-damaged skin, viscera and other organs in the absence of sensitization.

By “opioid analgesic” is meant an agent which binds to specific opioid receptors and agonises those receptors to produce reduced or absent pain perception without causing a loss of consciousness. Opioid analgesics include opiate alkaloids which may be isolated from opium and synthetic derivatives or analogues thereof.

In this specification “optionally substituted” is taken to mean that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocyclyloxy, heterocyclylamino, haloheterocyclyl, alkylsulphenyl, arylsulphenyl, carboalkoxy, carboaryloxy, mercapto, alkylthio, alkenylthio, alkynylthio, arylthio, benzylthio, heterocyclylthio, acylthio, cyano, nitro, sulfate and phosphate groups.

The term “pain” as used herein is given its broadest sense and includes an unpleasant sensory and emotional experience associated with actual or potential tissue damage, or described in terms of such damage and includes the more or less localised sensation of discomfort, distress, or agony, resulting from the stimulation of specialised nerve endings. There are many types of pain, including, but not limited to, lightning pains, phantom pains, shooting pains, acute pain, inflammatory pain, neuropathic pain, complex regional pain, neuralgia, neuropathy, and the like (Dorland's Illustrated Medical Dictionary, 28th Edition, W.B. Saunders Company, Philadelphia, Pa.). The goal of treatment of pain is to reduce the severity of pain perceived by a treatment subject.

By “pharmaceutically acceptable carrier” is meant a solid or liquid filler, diluent or encapsulating substance that may be safely used in topical, local or systemic administration.

The term “pharmaceutically compatible salt” as used herein refers to a salt which is toxicologically safe for human and animal administration. This salt may be selected from a group including, for example, hydrochlorides, hydrobromides, hydroiodides, sulphates, bisulphates, nitrates, citrates, tartrates, bitartrates, phosphates, malates, maleates, napsylates, fumarates, succinates, acetates, terephthalates, pamoates and pectinates.

The term “prodrug” is used in its broadest sense and encompasses those compounds that are converted in vivo to an opioid analgesic according to the invention. Such compounds would readily occur to those of skill in the art, and include, for example, compounds where a free hydroxy group is converted into an ester derivative. Prodrug forms of compounds may be utilised, for example, to improve bioavailability, mask unpleasant characteristics such as bitter taste, alter solubility for intravenous use, or to provide site-specific delivery of the compound.

By “slow-release nitric oxide donor” or “slow-release NO donor” is meant any substance that is converted or degraded or metabolised into, or provides a source of in vivo nitric oxide or NO over an extended period of time, thereby delivering a low concentration of nitric oxide into the blood stream. Suitably the slow-release nitric oxide donor is administered in an amount of 0.004 mmol/kg to 0.4 nmol/kg or in an amount such that nitric oxide is delivered at a rate of 0.0002 nmol/kg/hour to 2.0 nmol/kg/hour.

By “sub-analgesic amount” is meant an amount of analgesic which when administered alone, does not cause analgesia in a subject but produces analgesia when administered in combination with an effective amount of a slow-release nitric oxide donor.

The terms “subject” or “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment or prophylaxis for pain, especially moderate or severe pain. However, it will be understood that the aforementioned terms do not imply that symptoms are present.

The term “sustained-release formulation of nitric oxide donor” as used herein refers to a formulation of a nitric oxide donor which is adapted to release nitric oxide at a rate of 0.0002 mmol/kg/hour to 2.0 mmol/kg/hour or a range selected from 0.001 nmol/kg/hour to 1.0 nmol/kg/hour, 0.005 nmol/kg/hour to 1.0 nmol/kg/hour, 0.001 nmol/kg/hour to 0.5 nmol/kg/hour, 0.002 nmol/kg/hour to 0.2 nmol/kg/hour, 0.005 mmol/kg/hour to 0.1 mmol/kg/hour or 0.01 mmol/kg/hour to 0.05 mmol/kg/hour. The sustained release formulation may be any formulation capable of releasing nitric oxide at this rate. Preferred sustained release formulations are transdermal patches adapted to deliver 5 nmol to 500 nmol per 24 hours, especially 10 nmol to 100 nmol per 24 hours, more especially 20 nmol to 60 nmol per 24 hours, most especially about 50 mmol per 24 hours.

### Methods of Producing Analgesia

In one aspect, the present invention provides methods for producing analgesia in a subject. These methods generally comprise the administration of an effective amount of a nitric oxide donor and an opioid analgesic wherein the nitric oxide donor is a slow-release nitric oxide donor or a nitric oxide donor is a sustained release formulation adapted to deliver nitric oxide at a rate of 0.0002 mmol/kg/hour to 2.0 nmol/kg/hour. The nitric oxide donor is administered in an amount that increases the pain relieving potency of the opioid analgesic or the duration of analgesia produced by the opioid analgesic. The opioid analgesic is administered in an amount, that in combination with the nitric oxide donor, produces analgesia.

The slow-release nitric oxide donor is selected from any substance that is converted or degraded or metabolised into, or provides a source of, in vivo nitric oxide over an extended period of time. In one embodiment of the invention, the slow-release nitric oxide donor comprises a nitrato group coupled to a carrier compound by a linker. Preferred slow-release nitric oxide donors include those of Formula (I)

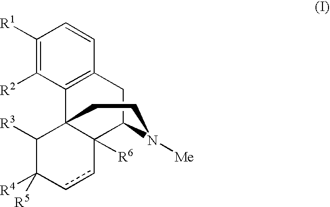

wherein R¹ is selected from OH, OC₁₋₆ alkyl, —O-A-X—NO₂,

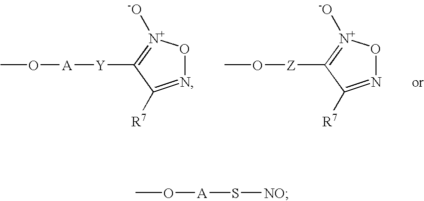

R² and R³ are each H or taken together are —O—;
R⁴ is H, OH, OC₁₋₆ alkyl, —O-A-X—NO₂,

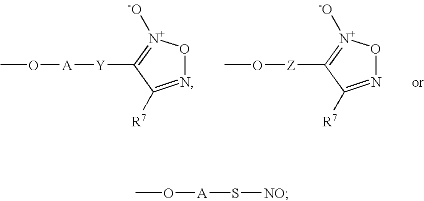

and R⁵ is H or R⁴ and R⁵ taken together form an oxo group;
R⁶ is selected from H, OH, OC₁₋₆ alkyl, —O-A-X—NO₂,

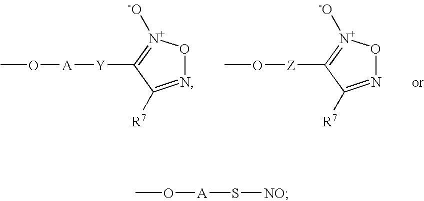

[symbol]

represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents

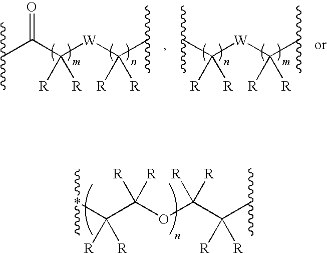

wherein W is absent or is selected from —O—, —S—, —NH—, —NC₁₋₆ alkyl,

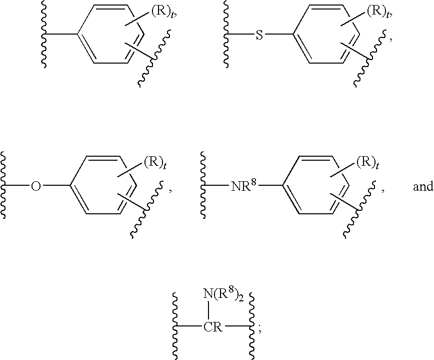

R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
m is 0 or an integer from 1 to 10;
n is an integer from 1 to 10; and
t is 0 or an integer from 1 to 4.
wherein at least one of R¹, R⁴ and R⁶ is —O-A-X—NO₂,

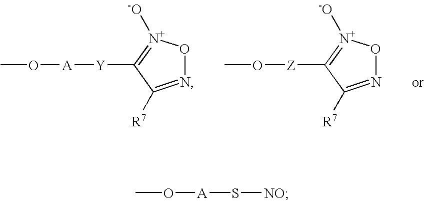

or a pharmaceutically acceptable salt thereof.

Exemplary groups of the formula —O-A-X—NO₂ include

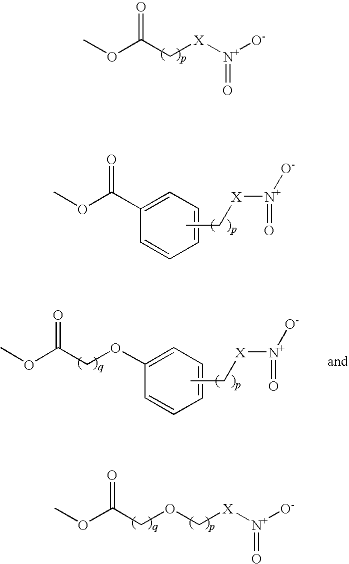

wherein X is S or O;
p is an integer from 1 to 10; and
q is 0 or an integer from 1 to 10.

Preferred compounds include those of Formula (II):

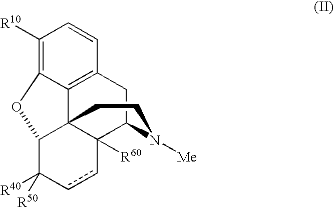

wherein R¹⁰ is selected from OH, OCH₃, —O-A-X—NO₂,

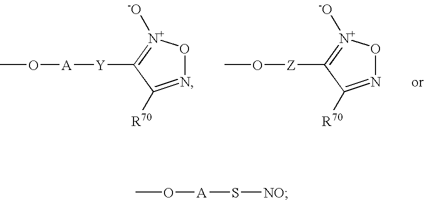

R⁴⁰ is selected from —O-A-X—NO₂,

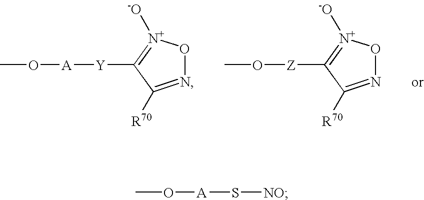

and R⁵⁰ is H or R⁴⁰ and R⁵⁰ taken together form an oxo group;
R⁶⁰ is selected from H or —O-A-X—NO₂,

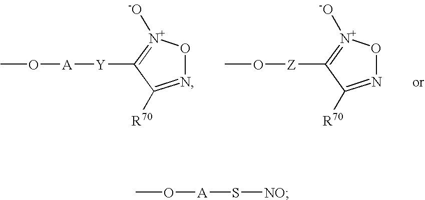

[symbol]

represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents

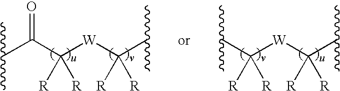

wherein W is absent or is selected from —O—, —S—, —NH—,

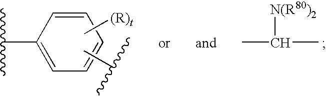

R⁷⁰ is selected from C₁₋₆ alkyl, C₁₋₆ alkoxy, C₁₋₄ alkylCO, C₁₋₆ alkylSO, C₁₋₆ alkylSO₂, phenyl, phenoxy, phenylSO, phenylSO₂, phenylCO, N(R⁸⁰)₂ and (R⁸⁰)₂NCO;
each R⁸⁰ is independently selected from H, C₁₋₆ alkyl, C₂₋₆ alkenyl, C₂₋₆ alkynyl or aryl;
each R is independently selected from H, C₁₋₄ alkyl, C₂₋₆ alkenyl, C₂₋₆ alkynyl, aryl, heterocyclyl, halo, hydroxy, C₁₋₄ alkoxy, aryloxy, C₂₋₆ alkenyloxy, heterocyclyloxy, thiol, C₁₋₆ alkylthiol, C₂₋₄ alkenylthiol, arylthiol, heterocyclylthiol, benzyl, benzyloxy, benzylthio, acyl, acyloxy, CO₂H, CO₂C₁₋₆ alkyl, SOC₁₋₆ alkyl, SO₂C₁₋₆ alkyl, SO₃H, SO₃C₁₋₆ alkyl, SONH₂, SONHC₁₋₆ alkyl, SON(C₁₋₆ alkyl)₂, SO₂NH₂, SO₂NHC₁₋₆ alkyl, SO₂N(C₁₋₆ alkyl)₂, CONH₂, CONHC₁₋₄ alkyl, CON(C₁₋₄ alkyl)₂, NH₂, NHC₁₋₄ alkyl, N(C₁₋₆ alkyl)₂, CN, CF₃ or NO₂;
u is 0 or an integer from 1 to 5;
v is an integer from 1 to 5; and
t is 0 or an integer from 1 to 4;
wherein at least one of R¹⁰, R⁴⁰ and R⁶⁰ is —O-A-X—NO₂,

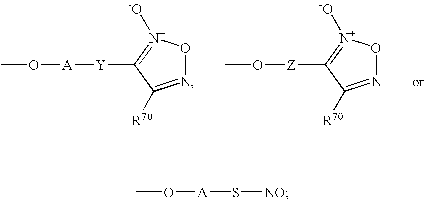

or a pharmaceutically acceptable salt thereof.

Examples of suitable slow-release nitric oxide donors include:

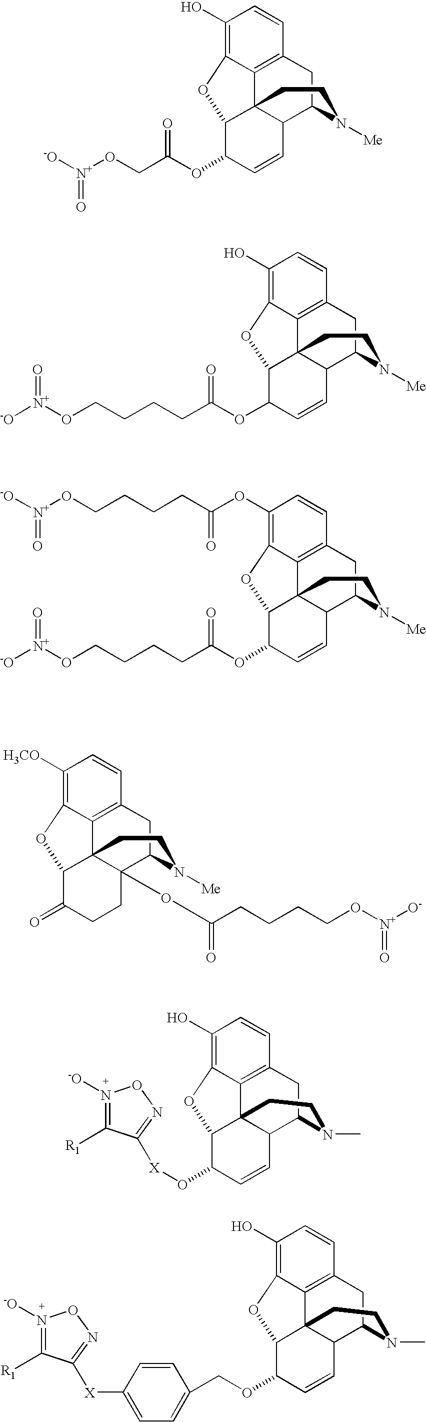

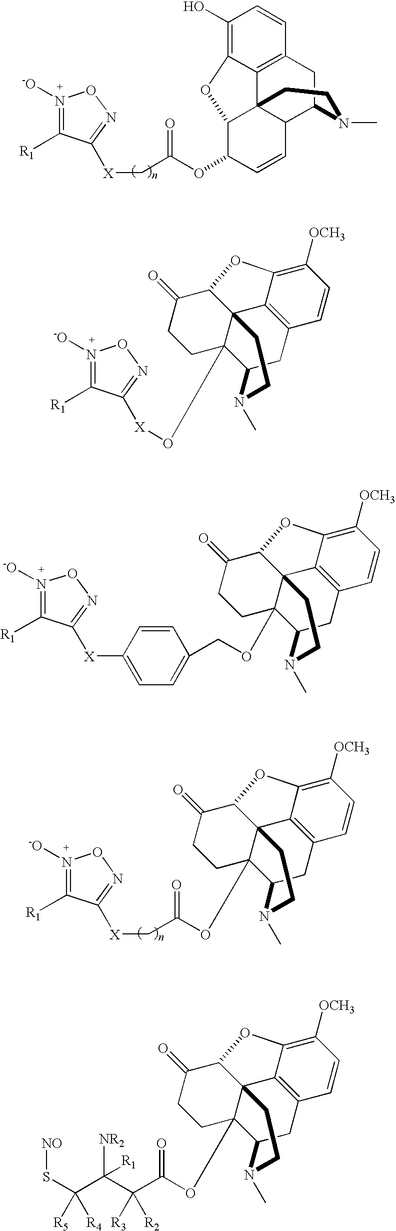

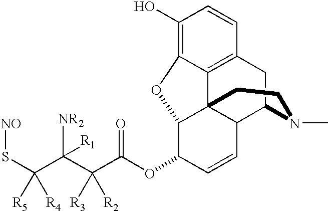

and their pharmaceutically acceptable salts.

Compounds of formula (I) may be prepared using synthetic methods known to those skilled in the art. Such methods may be found in texts such as Advanced Organic Chemistry, March J, 3^{rd} Edition, John Wiley and Sons, 1985 and Comprehensive Organic Transformations, R. C. Larock, VCH, 1989. It is also known in the art that functional groups may need protection and deprotection during synthetic processes. Suitable protection and deprotection methods may be found in texts such as Protective Groups in Organic Chemistry, T. W. Greene and P. G. M. Wuts, Wiley Interscience, 1999.

Compounds of formula (I) may be prepared by coupling a commercially available morphine compound or derivative or a morphine derivative prepared from a commercially available compound with a nitrato, furazanyl or —SNO containing linker. For example, a linker containing a free carboxylic acid or acid chloride and a nitrato, furazanyl or —SNO group may be coupled to a free hydroxy group of the morphine or morphine derivative by esterification methods well known in the art. For example, coupling may be achieved by treating the carboxylic acid and morphine derivative hydroxy group with a dehydrating agent such as dicyclohexylcarbodiimide (DCC). Alternatively, a linker containing a nitrato, furazanyl or —SNO group and a leaving group may be coupled with a free hydroxy to form an ether linkage.

In another aspect of the invention, there is provided compounds of formula (I), pharmaceutically acceptable salts thereof and compositions containing compounds of formula (I), pharmaceutically acceptable salts thereof and optionally an opioid analgesic.

An effective amount of a nitric oxide donor is one that is effective in enhancing or increasing the pain relieving potency or the duration of effect of the opioid analgesic. In one embodiment, the amount of slow-release nitric oxide donor that is administered as a bolus is in the range of 0.004 nmol/kg to 0.4 nmol/kg, preferably in a range selected from 0.005 mmol/kg to 0.3 nmol/kg, 0.006 mmol/kg to 0.2 nmol/kg, 0.007 nmol/kg to 0.1 nmol/kg, 0.008 nmol/kg to 0.09 nm no/kg, 0.009 nmol/kg to 0.08 nmol/kg, 0.01 nmol/kg to 0.07 nmol/kg, 0.02 mmol/kg to 0.06 nmol/kg, and especially 0.03 nmol/kg to 0.05 nmol/kg. An especially preferred amount is 0.04 nmol/kg. In another embodiment, the nitric oxide donor is formulated in a sustained release formulation adapted to release nitric oxide at a rate of 0.0002 nmol/kg/hour to 2.0 nmol/kg/hour or in a range selected from 0.001 nmol/kg/hour to 1.0 nmol/kg/hour, 0.005 nmol/kg/hour to 1.0 mmol/kg/hour, 0.001 nmol/kg/hour to 0.5 nmol/kg/hour, 0.002 nmol/kg/hour to 0.2 nmol/kg/hour, 0.005 nmol/kg/hour to 0.1 nmol/kg/hour, or 0.01 mmol/kg/hour to 0.05 nmol/kg/hour. A particularly preferred embodiment is a transdermal patch adapted to release 5 mmol to 500 nmol especially 10 nmol to 100 nmol, more especially 20 nmol to 60 mmol and even more especially about 50 nmol per 24 hours.

Without wishing to be bound by any one theory or mode of operation, it is postulated that the slow-release of NO from the nitric oxide donor allows maintenance of a low concentration of NO in the blood stream. Slow release of NO appears to favour the formation of NO⁺ and possibly NO⁻, which are thought to be important in the endogenous formation of nitrosothiols. Slow release of NO does not favour formation of NO⁻ which readily reacts with superoxide (O₂⁻) to produce the potent neurotoxin, peroxynitrite (ONOO⁻).

It is postulated that endogenous nitrosothiols facilitate the nitrosylation of the N-methyl-D aspartate receptor (NMDA receptor) thereby attenuating the activation of this receptor resulting in the attenuation of the pain response. In one embodiment, the effective amount of a slow-release nitric oxide donor or sustained release formulation of nitric oxide donor is one that is effective in releasing NO in the form of NO⁺ or NO⁻. In yet another embodiment, the effective amount of slow-release nitric oxide donor or sustained release formulation of nitric oxide donor is one that enhances endogenous production of nitrosothiols, or reduces endogenous production of peroxynitrite or causes more endogenous production of nitrosothiols than endogenous production of peroxynitrite. The endogenous production of peroxynitrite may be assessed by measurement of nitrotyrosine in a biological fluid.

In yet another embodiment, the effective amount of a nitric oxide donor is one that increases the ratio of nitrosothiol concentration:peroxynitrite concentration in a biological fluid by a factor of at least 1.1 when compared to the ratio of nitrosothiol concentration peroxynitrite concentration that is observed upon administration of a sustained release formulation of nitroglycerine which delivers 5 mg of nitroglycerine per 24 hours. In preferred embodiments, the ratio of nitrosothiol concentration:peroxynitrite concentration increases by a factor of between 1.25 and 100, preferably 1.25 and 500, more preferably between 1.25 and 1000, when compared with the ratio of nitrosothiol concentration:peroxynitrite concentration observed upon administration of a sustained release formulation of nitroglycerine. It is especially preferred when this ratio increases by a factor of between 1.25 and 5, 1.25 and 10, 1.25 and 20, 1.25 and 30, 1.25 and 40, 1.25 and 50, 1.25 and 60, 1.25 and 70, 1.25 and 80, 1.25 and 90 or 1.25 and 100.

It has been suggested that in biological systems, nitric oxide is stabilised by nitrosothiol formation, which preserves its biological activity. Nitrosothiols have significantly longer half-lives in vivo than nitric oxide and also act as potent platelet-inhibitory and vasodilatory agents. The predominant nitrosothiol in mammalian plasma is nitrosoalbumin (AlbSNO) which then transfers nitric oxide to low molecular weight thiols such as glutathione (GSH) or cysteine (CySH) to form GSNO or CySNO respectively. The low molecular weight nitrosothiols can then diffuse to the required site of action and release nitric oxide.

The level of nitrosothiols, such as AlbSNO, GSNO and CySNO, in the blood may be determined using a suitable assay. The level of peroxynitrite (ONOO⁻) formed by reaction of nitric oxide (NO) with superoxide (O₂⁻) can be determined by monitoring the level of nitrotyrosine in the biological fluid. Amounts of nitrosothiols, such as S-nitrosoglutathione (GSNO) and S-nitrosocysteine (CySNO), and nitrotyrosine may be determined simultaneously in a biological fluid, such as plasma ultrafiltrate, using a high performance liquid chromatography-electrospray ionisation-tandem mass spectrometry (HPLC-ESI-MS-MS) assay and deuterated analogues of cysteine, glutathione and nitrotyrosine as internal standards. Chromatographic separation is achieved with an Agilent Zorbax C18 2.1×50 mm column type using a mobile phase comprising Components A (0.1% v/v formic acid) and Components B (0.1% formic acid in 90:10 methanol water). This method is adapted from the methods of Kluge et. al., (1997), J. Neurochem., 69:2599-2607 and Orhan et. al., (2004), J. Chrom. B., 799:245-254.

The opioid analgesic may be any opioid compound having analgesic activity. In a preferred embodiment, the opioid analgesic is selected from morphine, methadone, fentanyl, sufentanil, alfentanil, hydromorphone, oxymorphone, oxycodone, codeine, hydrocodeine, hydrocodone, levorphanol, meperidine, heroin, morphine-6-glucuronide, levallorphan, 6-monoacetylmorphine and tramadol.

The dose of active compounds administered to a patient should be sufficient to achieve a beneficial response in the patient over time such as a reduction in, or relief from, pain. The quantity of the pharmaceutically active compound(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the production of analgesia, the physician may evaluate severity of the pain symptoms associated with nociceptive or inflammatory pain conditions and in the amount of opioid analgesic, may consider whether the patient is opioid analgesic naïve or whether previous long term exposure to an opioid analgesic has occurred. In any event, those of skill in the art may readily determine suitable dosages of the nitric oxide donors and/or the opioid analgesic of the invention without undue experimentation.

An effective amount of opioid analgesic may be an amount which is the recommended dosage for opioid naïve patients or for patients tolerant to analgesic effects of opioids. For example, in a morphine naïve adult patient, a standard dosage is 5-20 mg if delivered by intramuscular or subcutaneous injection, or 2.5-15 mg if delivered by intravenous injection. Morphine may also be administered in an oral immediate release tablet or capsule in a dosage of 10-30 mg or in an oral sustained release dosage form of 40 mg or 20 mg. Morphine may also be administered to a morphine naïve adult patient by epidural administration (5 mg), intrathecal administration (0.2-1 mg) or by intracerebroventricular administration (0.1-1 mg). Dosages of morphine suitable for administration to children include 0.1-0.2 mg/kg to a maximum of 15 mg by intramuscular or subcutaneous injection or with caution 0.05-0.1 mg/kg incrementally over 5-15 minutes if titrated intravenously. Although the above dosages for intramuscular or subcutaneous injection or oral immediate release tablets or capsules are normally provided at a frequency of every 4-6 hours, in combination with a nitric oxide donor according to the invention, the frequency of dosing may be extended to every 5-7 hours, 6-8 hours, 7-9 hours, 8-10 hours, 9-11 hours or 11-12 hours. Although the above dosage forms for oral sustained release formulations are normally provided at a frequency of 40 mg/24 hours or 20 mg/12 hours, these formulations may, in combination with a nitric oxide donor according to the invention, be provided at longer intervals, such as 25, 26, 27, 28, 29, 30, 31, 32, 33, 34, 35 or 36 hours or 13, 14, 15, 16, 17 or 18 hours. Standard doses given above for epidural, intrathecal or intracerebroventricular administration are normally provided at a frequency of every 24 hours. However, in combination with the nitric oxide donor according to the present invention, the frequency of dosing may be extended to, for example, 25, 26, 27, 28, 29, 30, 31, 32, 33, 34, 35 or 36 hour intervals.

Standard oxycodone dosages for opioid naïve adult patients include 1-10 mg by intravenous injection or 1-10 mg by intramuscular or subcutaneous injection. Oral administration may be by immediate release tablets in a dosage of 5-10 mg or in a sustained release oral dosage form of 10 mg. Oxycodone dosages may also be administered in 30 mg by rectal suppository. Although the above oxycodone dosages for intravenous, intramuscular or subcutaneous injection or oral immediate release tablets are normally provided every 4-6 hours, in combination with the nitric oxide donor of the present invention, the frequency of dosing may be extended, for example, to every 5-7 hours, 6-8 hours, 7-9 hours, 8-10 hours, 9-11 hours or 11-12 hours. Sustained release oral dosages of oxycodone are normally provided every 12 hours, however, in combination with a nitric oxide donor according to the present invention, this frequency may be extended for example, to every 13 hours, 14 hours, 15 hours, 16 hours, 17 hours or 18 hours. The rectal suppository form of oxycodone is normally provided at a frequency of every 6-8 hours, however, in combination with a nitric oxide donor according to the present invention, this frequency of dosing may be extended to, for example, every 7-9 hours, 8-10 hours, 9-11 hours, 10-12 hours, 11-13 hours, 12-14 hours, 13-15 hours or 14-16 hours.

Standard hydromorphone dosages for the production of analgesia in opioid-naïve patients include an oral dosage of 2-4 mg, 1-2 mg by intramuscular or subcutaneous injection, or 0.5-1.0 mg by intravenous injection delivered over 2-3 minutes. The frequency of administration of the oral dosage form is usually every 4 hours, however, in combination with the nitric oxide donor according to the present invention, the frequency of dosing may be extended to, for example, every 5 hours, 6 hours, 7 hours, 8 hours, 9 hours or 10 hours. The frequency of dosing of the intramuscular or subcutaneous injection dosage forms is usually every 2 hours. However, in combination with a nitric oxide donor according to the present invention, this dosing frequency may be extended to, for example, every 3 hours, 4 hours, 5 hours, 6 hours, 7 hours or 8 hours. Suitable dosages for children include oral dosages of 60 μg/kg or 15 μg/kg if delivered by intramuscular, subcutaneous or intravenous injection. The frequency of dosing for both oral and injectable forms of hydromorphone in children is usually every 3-4 hours. However, in combination with a nitric oxide donor according to the present invention, the frequency of dosing may be extended to, for example, every 4-5 hours, 5-6 hours, 6-7 hours, 7-8 hours, 8-9 hours or 9-10 hours.

Suitable doses of fentanyl for the production of analgesia in opioid-naïve adult patients include 50-100 μg administered intramuscularly 30-60 minutes prior to surgery and 50-100 μg administered intramuscularly post-operatively as needed. Post-operative fentanyl is often delivered every 1-2 hours, however, in combination with the nitric oxide donor according to the present invention, the frequency of delivery may be extended to, for example, every 2-3 hours, 3-4 hours, 4-5 hours, 5-6 hours, 6-7 hours or 7-8 hours. Fentanyl may also be delivered by transdermal patch at a dosage of 25 μg/hour.

Alternatively, the opioid analgesic may be administered in a sub-analgesic amount, which when administered alone, does not cause analgesia in a subject, however, when administered in combination with an effective amount of a slow-release nitric oxide donor, results in analgesia. For example, the sub-analgesic amount may be an ED₁₀ to ED₉₀ amount, which corresponds to a dose which is effective to produce an analgesic response in 10 to 90% of patients or subjects. Preferably, the sub-analgesic amount corresponds to one of an ED₁₀ to ED₈₀ amount, an ED₁₀ to an ED₇₀ amount, an ED₁₀ to an ED₆₀ amount, an ED₁₀ to an ED₅₀ amount, an ED₁₀ to an ED₄₆ amount and especially an ED₁₀ to an ED₃₀ amount. An especially preferred effective sub-analgesic amount is an ED₂₀ amount.

The methods of producing analgesia are suitable for use in the treatment of pain, particularly moderate to severe pain. The treatment is suitable for use in any situation where the use of an opioid analgesic would normally be indicated. For example, the treatment is suitable for use in the relief of nociceptive pain such as moderate to severe cancer pain, the alleviation of moderate to severe pain in the post-surgical setting and the relief of pain following physical trauma such as soft tissue injury after an accident, pain associated with cardiac infarction and relief of inflammatory pain such as arthritic pain.

When the amount of opioid analgesic administered is a standard or analgesic amount (ED₁₀₀), and is administered in combination with an effective amount of nitric oxide donor, the duration of analgesic effect may be longer than that experienced when the same amount of opioid analgesic is administered alone. This results in less frequent dosing of a subject with the opioid analgesic and therefore fewer side effects are experienced and/or the side effects are of lesser severity.

When the amount of opioid analgesic administered is a sub-analgesic amount, and is administered in combination with an effective amount of a nitric oxide donor, the analgesic effect experienced is of similar potency and duration as that experienced when a dosage 1.5 to 5 times greater, for example 3 times greater, is administered. This results in administration of much less opioid analgesic being administered in any one dose and therefore fewer side effects are experienced and/or the side effects are of lesser severity. For example, a reduction in any one or more of the following: less allergic reactions such as no or reduced difficulty breathing, swelling of lips, tongue, face and/or throat, or hives; no or reduced respiratory depression, less seizures or seizures of reduced severity; less cold, clammy skin, reduced weakness, no or reduced dizziness, reduced likelihood of unconsciousness, reduced or no sedation, reduced or no nausea, reduced or no vomiting or dry mouth, a reduction in loss of appetite, reduced or no constipation, reduced or no tiredness, reduced or no lightheadedness, reduced or no muscle twitching, reduced or no sweating, reduced or no pruritis, reduced or no urinary retention, and a reduction in loss of libido. There may also be a reduced likelihood of development of opioid analgesic tolerance or dependence.

The effect of the combination of slow-release nitric oxide donor and opioid analgesic may be examined using one or more of the published models of pain/nociception known in the art. The analgesic activity may be evaluated using methods known in the art, such as the Tail-flick Test (D'Amour et. al., 1941, J. Pharmacol. Exp. Ther. 72:74-79), the hotplate test (Eddy and Leimbach, 1953, J. Pharmocol. Exp. Ther., 107:385-93), the paw pressure test (Randall and Selitto, 1957, Arch. Int. Pharmacodyn., 111:409-414), the paw thermal test (Hargreaves et. al., 1998, Pain, 32:77-88) and the Brennan model of post-surgical pain (Brennan et al., 1996, Pain, 64:493-501).

While the nitric oxide donor and opioid analgesic may be administered simultaneously in a single composition or simultaneously or sequentially in separate administration as neat or undiluted compounds, it is more common to administer the compounds in a pharmaceutical composition. Suitable compositions include an effective amount of active agent to achieve its purpose and a pharmaceutically acceptable carrier, diluent or excipient.

In one embodiment, and dependent on the intended mode of administration, the nitric oxide donor-containing compositions will generally contain about 0.1% to 90%, about 0.5% to 50%, or about 1% to about 25%, by weight of nitric oxide donor, the remainder being suitable pharmaceutical carriers and/or diluents etc and optionally an opioid analgesic. The dosage of the nitric oxide donor can depend on a variety of factors, such as the individual nitric oxide donor, mode of administration, the species of the affected subject, age and/or individual condition.

In another embodiment, and dependent on the intended mode of administration, the opioid analgesic-containing compositions will generally contain about 0.1% to 90%, about 0.5% to 50%, or about 1% to about 25%, by weight of opioid analgesic, the remainder being suitable pharmaceutical carriers and/or diluents etc and optionally a nitric oxide donor.

Depending on the specific pain being treated, the active compounds may be formulated and administered systemically, topically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, epidural, direct intraventricular, intravenous, intraperitoneal, inhalational, intranasal, or intraocular injections. For injection, the therapeutic agents of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Alternatively, the compositions of the invention can be formulated for local or topical administration. In this instance, the subject compositions may be formulated in any suitable manner, including, but not limited to, creams, gels, oils, ointments, solutions and suppositories. Such topical compositions may include a penetration enhancer such as benzalkonium chloride, digitonin, dihydrocytochalasin B, capric acid, increasing pH from 7.0 to 8.0. Penetration enhancers which are directed to enhancing penetration of the active compounds through the epidermis are advantageous in this regard. Alternatively, the topical compositions may include liposomes in which the active compounds of the invention are encapsulated.

The opioid analgesic and the slow-release nitric oxide donor may be formulated in a single composition, or may be formulated separately for simultaneous or sequential delivery by the same or different modes of administration. For example, the opioid analgesic may be formulated for oral delivery while the nitric oxide donor is formulated to be delivered by a transdermal patch, or the opioid analgesic may be formulated for parenteral administration while the slow-release nitric oxide donor is formulated for oral delivery, or both the opioid analgesic and the slow-release nitric oxide donor may be formulated in a single composition or separate compositions for oral delivery, or the nitric oxide donor may be formulated for transdermal delivery while the opioid analgesic is formulated for parenteral administration. Other combinations of modes of delivery could be readily determined by those skilled in the art.

The compositions of this invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the active compounds of the present invention can be formulated readily using pharmaceutically acceptable carriers well known in the art into dosages suitable for oral administration, which is also preferred for the practice of the present invention. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, aid/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceuticals which can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the active compounds of the invention may also include injecting or implanting controlled releasing devices designed specifically for this purpose or other forms of implants modified to act additionally in this fashion. Controlled release of an active compound of the invention may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be achieved by using other polymer matrices, liposomes and/or microspheres. Controlled release may also be achieved using a transdermal patch, particularly a transdermal patch in which the rate of release of the nitric oxide donor is controlled by a co-polymer release membrane or in which the nitric oxide donor is embedded in a biodegradable matrix that dissolves at a known rate. Transdermal patches which allow slow and sustained delivery of a drug at a known rate are known in the art.

The active compounds of the invention may be administered over a period of hours, days, weeks, or months, depending on several factors, including the severity of the pain being treated, whether the pain is chronic or whether a recurrence of the pain is considered likely, etc. The administration may be constant, e.g., constant infusion over a period of hours, days, weeks, months, etc. Alternatively, the administration may be intermittent, e.g., active compounds may be administered once a day over a period of days, once an hour over a period of hours, or any other such schedule as deemed suitable.

The compositions of the present invention may also be administered to the respiratory tract as a nasal or pulmonary inhalation aerosol or solution for a nebuliser, or as a microfine powder for insufflation, alone or in combination with an inert carrier such as lactose, or with other pharmaceutically acceptable excipients. In such a case, the particles of the formulation may advantageously have diameters of less than 50 micrometers, suitably less than 10 micrometers.

In order that the invention may be readily understood and put into practical effect, particular preferred embodiments will now be described by way of the following non-limiting examples.

### EXAMPLES

### Example 1

### Preparation of Morphine Conjugate (2)

### Nitratoacetic Acid (1)

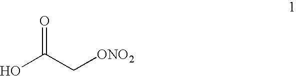

To a solution of chloroacetic acid (1.89 g, 20 mmol) in 5 ml of dry acetone was added a solution of NaI (30 mmol, 4.5 g) in 10 ml of dry acetone and the mixture was heated under reflux for 1 hour. Then the solvent was removed and the residue was treated with 10 ml of water. The mixture was extracted with diethyl ether (3×50 ml) and the combined organic phases were washed with brine, sat. Na₂S₂O₃ solution, dried over Na₂SO₄ and the solvent was removed in vacuum yielding 2.8 g of a yellow solid, that was used without further purification.

The solid was dissolved in 10 ml of dry acetonitrile and a solution of AgNO₃ (5.1 g, 30 mmol) in 20 ml of acetonitrile was added. The mixture was stirred at room temperature overnight and a yellow precipate formed. To this mixture was added ±0 ml of brine and the mixture was filtered off. The filtrate was extracted with diethyl ether (3×50 ml), the combined organic phases were washed with brine, dried over Na₂SO₄ and the solvent was removed in vacuum affording a yellow solid (1.45 g, 12 mmol, 60%).

¹H NMR (CDCl₃, 200 MHz): δ=10.44 (bs, 1H, COOH), 4.98 (s, 2H, 2-H).

### Morphine Conjugate (2)

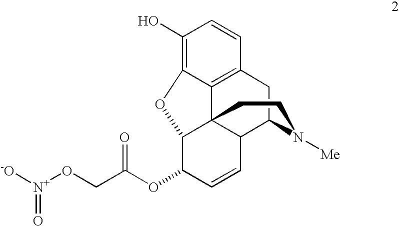

Nitratoacetic acid (64 mg, 0.53 mmol) was dissolved in 40 ml of anhydrous chloroform and freshly prepared free morphine base (150 mg, 0.53 mmol) and dicyclohexylcarbodiimide (109 mg, 0.53 mmol) were added under an argon atmosphere. The mixture was heated to 70° C. for 10 hours. Then an additional portion of nitratoacetic acid (64 mg) and dicyclohexylcarbodiimide (109 mg) were added. Heating was continued for 6 hours. Then the mixture was cooled to room temperature and the solvent was removed in vacuum. The residue was treated with water (20 ml) and stirred for 20 minutes. Then the precipate was filtered off and washed with water (2×10 ml). The aqueous solution was concentrated in vacuum affording 120 mg (0.31 mmol, 59%) of (2) as a brown solid.

### Example 2

### Preparation of Morphine-Oxide Conjugate 5

### (a) Morphine

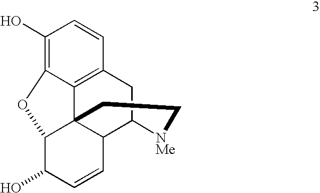

Morphine hydrochloride trihydrate (1.5 g) was dissolved in the minimum amount of water (RO type) (˜20 mL) and to this was added enough saturated sodium hydrogen carbonate to precipitate morphine. Morphine 3 was collected by vacuum filtration and washed with distilled water (20 mL) followed by small amounts of cold diethyl ether (5 mL). The white solid, protected from light with aluminium foil, was placed under high vacuum (0.01 mmHg) for 3 hours.

### (b) 5-Nitratovaleric Acid

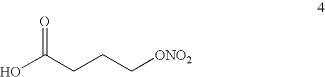

The titled compound was prepared following the procedure of EP 0 984 012 A2 (K. M. Lundy, M. T. Clark). Briefly, silver nitrate (23.48 g, 0.153 mol) was pre-dried under high vacuum (0:01 mmHg) and subsequently dissolved in anhydrous acetonitrile (70 mL) under an argon atmosphere. The solution was warmed to 50° C. and 5-bromovaleric acid (5 g, 0.028 mol) [dissolved in anhydrous acetonitrile (3 mL)] added quickly via syringe. A precipitate formed instantaneously. The mixture was then heated at 80° C. for 20 mins. On cooling the precipitate (AgBr) was removed by filtration. The filtrate was concentrated and the residue partitioned between ethyl acetate and water. The ethyl acetate layer was then washed with water, dried (Na₂SO₄), concentrated and further dried under vacuum (0.01 mm Hg). The titled compound was used without further purification.

### (c) Morphine NO Donor

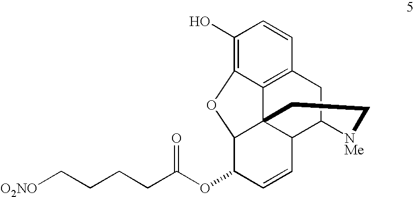

Freshly prepared morphine 3 (500 mg, 1.75 mmol), dicyclohexylcarbodiimide (362 mg, 1.75 mmol), and 5-nitratovaleric acid 4 (286 mg, 1.75 mmol) were dissolved in anhydrous chloroform (90 mL) under an argon atmosphere. The mixture was refluxed for 12 hours and allowed to cool. Additional dicyclohexylcarbodiimide (362 mg, 1.75 mmol), and 5-nitratovaleric acid (286 mg, 1.75 mmol) were added and refluxing continued for 6 hours. On cooling the solvent was removed in vacuo and the residue dissolved in a solution of warmed ethyl acetate/methanol (6:4) (˜5 mL) and filtered to remove N,N-dicyclohexylurea. The filtrate is concentrated and subjected to column chromatography (ethyl acetate/methanol; 6:4) on silica gel which affords morphine derivative 5 as a pale yellow solid (600 mg, 80%). ¹H n.m.r (200 MHz) 1.70-1.95 (m, 5H), 2.07 (dt, 1H), 2.22-2.38 (m, 2H), 2.4° (s, 3H), 2.54-2.73 (m, 3H), 3.05 (d, 1H), 3.35 (bs, OH), 3.33-3.40 (m, 2H), 4.08-4.20 (m, 1H), 4.40-4.55 (m, 2H), 4.90 (d, 1H), 5.20-5.34 (m, 1H), 5.67-5.78 (m, 1H), 6.65 (dd, 2H). Mass spectrum m/z (EI) 430 (M^{+•}, 27%), 384 (1), 366 (1), 354 (18), 326 (1), 285 (100), 268 (10), 215 (18), 174 (8), 162 (21), 124 (13), 94 (6).

### Tartaric Acid Salt of Morphine NO Donor 5

The above compound 5 (300 mg, 0.697 mmol) was suspended in water (RO type) (15 mL) and tartaric acid (1105 mg, 0.697 mmol) added. The mixture was stirred for 30 mins before addition of dimethylsulfoxide (AR grade) (15 mL). The resulting solution was stored at −20° C.

### Example 3

### Preparation of Morphine-Nitric Oxide Conjugate 7

### 5-Nitratovaleroyl Chloride

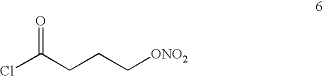

The titled compound was prepared following the procedure of EP 0 984 012 A2 (K. M. Lundy, M. T. Clark). Briefly, 5-nitratovaleric acid (13.34 g, 0.082 mol) was pre-dried under high vacuum (0.01 mmHg) and subsequently dissolved in anhydrous dichloromethane (200 mL) under an argon atmosphere. To this was added phosphorous pentachloride (17.03 g, 0.082 mol) portionwise over 2 mins. The mixture was stirred for 15 hours at room temperature. The solvent and excess hydrochloric acid was removed in vacuo and the residue dissolved in anhydrous toluene. The toluene was then 90% removed by distillation under argon at atmospheric pressure. [Warning: distillation must not be allowed to completely remove toluene as this will result in spontaneous explosive decomposition] Toluene is essential for removal of phosphorous oxy chloride. The toluene acid chloride mixture was used without further purification.

### Morphine NO Donor

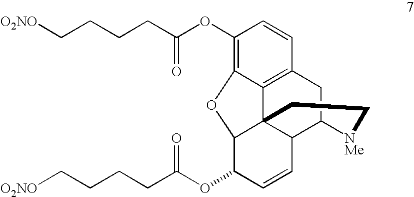

Morphine hydrochloride trihydrate (50 mg, 0.133 mmol) and 5-nitratovaleroyl chloride 6 (169 mg, 0.931 mmol) were heated together neat at 135° C. for 7 mins which affords a homogeneous mixture. On cooling the liquid is diluted with dichloromethane (10 mL) and transferred to a separatory funnel containing saturated sodium hydrogen carbonate solution (20 mL). After several washings the organic layer was dried (Na₂SO₄) and evaporated. The residue was subjected to column chromatography (ethyl acetate/methanol, gradient) on silica affording the morphine NO Donor 7 as a brown oil. ¹H n.m.r (200 MHz) 1.60-2.01 (m, 12H), 2.25-2.71 (m, 4H), 2.65 (s, 3H), 2.89-3.28 (m, 3H), 3.65-3.75 (m, 1H), 4.35-4.55 (m, 4H), 5.09-5.25 (m, 2H), 5.32-5.45 (m, 1H), 5.60-5.71 (m, 1H), 6.55-6.85 (m, 2H). Mass spectrum m/z (EI) 575 (M^{+•}, 6%), 548 (1), 530 (1), 503 (1), 472 (1), 454 (1), 430 (1), 403 (1), 385 (1), 354 (1), 285 (20), 268 (60), 215 (22), 162 (20), 146 (13), 124 (13), 100 (24), 81 (19), 42 (100).

### Example 4

### Preparation of Oxycodone-Nitric Oxide Conjugate 9

### Oxycodone

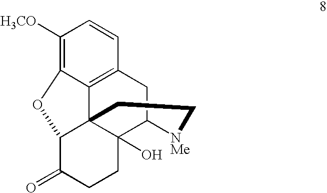

Oxycodone hydrochloride (1.5 g) was dissolved in the minimum amount of water (RO type) (˜20 mL) and to this was added enough saturated sodium hydrogen carbonate to raise the pH of the solution to about 11 and to precipitate oxycodone. Oxycodone 8 was collected by vacuum filtration and washed with distilled water (20 mL) followed by small amounts of cold diethyl ether (5 mL). The white solid, protected from light with aluminium foil, was placed under high vacuum (0.01 mm Hg) for 3 hours.

### Oxycodone NO Donor

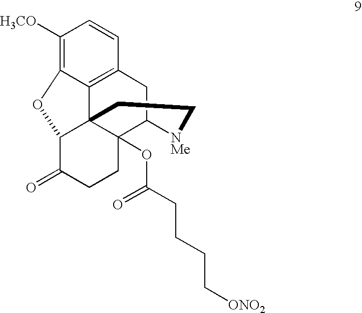

Freshly prepared oxycodone 8 (500 mg, 1.59 mmol), dicyclohexylcarbodiimide (327 mg 1.59 mmol), and 5-nitratovaleric acid 4 (259 mg, 1.59 mmol) were dissolved in anhydrous chloroform (90 mL) under an argon atmosphere. The mixture was refluxed for 12 hours and allowed to cool. Additional dicyclohexylcarbodiimide (327 mg, 1.59 mmol), and 5-nitratovaleric acid (259 mg, 1.59 mmol) were added and refluxing continued for 6 hours. On cooling the solvent was removed in vacuo and the residue dissolved in a solution of warmed ethyl acetate (˜5 mL) and filtered to remove N,N-dicyclohexylurea. The filtrate was concentrated and subjected to column chromatography (ethyl acetate/dichloromethane; gradient) on silica gel which affords derivative 9 as a pale yellow solid.

### Tartaric Acid Salt of Oxycodone—Nitric Oxide Conjugate

The above compound 9 (300 mg, 0.651 mmol) was suspended in water (RO type) (15 mL) and tartaric acid (98 mg, 0.651 mmol) added. The mixture was stirred for 30 mins before addition of dimethylsulfoxide (AR grade) (15 mL). The resulting solution was stored at −20° C.

### Example 5

### Assessment of Antinociceptive Response Using the Tail Flick Latency Test

### Animals

Adult male Sprague-Dawley rats (225-250 g) were purchased from the Herston Medical Research Centre, The University of Queensland (Brisbane, Australia) and adult male Sprague-Dawley rats were purchased from the Central Animal Breeding House, The University of Queensland (Brisbane, Australia). Rats were housed in a temperature controlled environment (21±2° C.) with a 12 h/12 h light/dark cycle. Food and water were available ad libitum. Ethical approval for this study was obtained from the Animal Experimentation Ethics Committee of The University of Queensland.

### Reagents and Materials

Morphine hydrochloride powder (B.P.) was purchased from the Royal Brisbane Hospital Pharmacy (Brisbane, Australia). Normal saline ampoules were obtained from Delta West Pty Ltd (Perth, Australia) and medical grade CO₂ and O₂ were purchased from BOC Gases Ltd (Brisbane, Australia),

### Preparation of Stock Solutions

Stock solutions of compound (2) and morphine HCl were prepared in normal saline to achieve final concentrations of 10 μmol/mL and 35 μmol/mL, respectively. Aliquots of stock solutions were stored at −20° C. until required.

### Drug Administration

Whilst lightly anaesthetized with a 50%:50% mixture of O₂:CO₂, groups of rats were administered either single bolus doses of (2) (0.04 nmol/kg), morphine (2.8 μmol/kg [≈ED₂₀], 10 μmol/kg), or a combination of the two, via the subcutaneous (s.c.) route. Antinociception was quantified using the tail-flick test over a 6 h post-dosing period.

### Assessment of Antinociceptive Responses Using the Tail Flick Latency Test

The tail flick latency test was used to quantify antinociception in rats (D'Amour and Smith, 1941, J. Pharmacol. Exp. and Ther., 72:74-79). This involved application of a noxious thermal stimulus to the lower third of the ventral surface of the rat's tail. Prior to drug administration, baseline tail flick responses were measured at 5 min intervals until three baseline latency values were obtained that were within ±1s (only three readings were required in most rats and no more than five readings were required in any rats). A maximum tail flick latency of 9.0 s was used to minimize tissue damage to the rats' tails. Tail flick testing was performed at the following times: pre-dose, 0.08, 0.25, 0.5, 0.45, 1, 1.5, 2, 3, 4, 6 h post-dosing.

### Data Analysis

Raw tail flick latency values obtained from each rat were normalized by conversion to a percentage of the maximum possible effect (% MPE; Brady and Holtzmann, Pharmacol. Exp. Ther., 1982, 222:190-7):

$$\% M P E = \frac{Postdrug Response - Predrug Response \times 100}{Maximum Response {(}^{*} ) - Predrug Response}$$

Where the maximum tail flick latency is 9.0 s

Levels of antinociception (% MPE values) were plotted against time to produce response (% MPE) versus time curves.

The area under the % MPE versus time curve (% MPE AUC) was calculated using trapezoidal integration.

### Statistical Analysis

The Mann-Whitney test was used to compare differences in the normalized % MPE AUC values between treatment groups. Statistical analysis was undertaken using the GraphPad Prism™ (version 3) software package, and the statistical significance criterion was P<0.05.

### Antinociceptive Potency of Compound 2 to a Noxious Thermal Stimulus

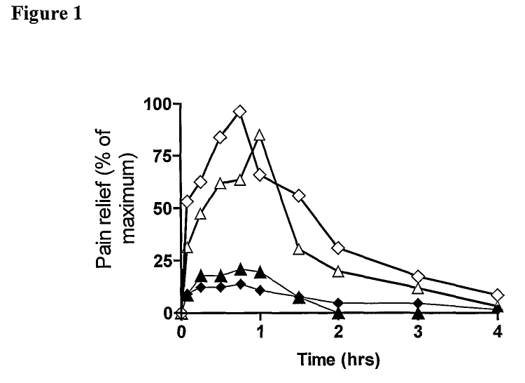

The antinociceptive potency of compound 2 (from Example 1) alone and in combination with morphine was assessed using an acute pain model in rats involving the application of a noxious thermal (heat) stimulus to the tail (tail-flick-test) of naïve rats (FIG. 1). Following administration of small s.c. bolus doses of 2 (0.04 nmol/kg) in combination with the ˜ED₂₀ of morphine (2.8 μmol/kg), maximum pain relief (antinociception) was produced at 45 min post-dosing and the duration of action was 34 hours. By contrast, when single s.c. bolus doses of 2 (0.04 nmol/kg) or morphine (2.8 μmol/kg) were administered alone to rats, the peak levels of antinociception were very low, viz 14% and 21% of the maximum possible effect (% NPE) respectively, and the corresponding durations of action were relatively short in the range 1.5-2 hours (FIG. 1). These findings collectively show that when this low dose of 2 (0.04 mmol/kg) is co-administered with morphine, there is a large increase in the extent and duration of antinociception and this occurred without a concomitant increase in CNS side-effects such as sedation.

The disclosure of every patent, patent application and publication cited herein is hereby incorporated by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-31. (canceled)

32. A method of producing analgesia in a subject comprising administering to the subject an effective amount of a nitric oxide donor and an effective amount of an opioid analgesic, wherein the effective amount of nitric oxide donor delivers nitric oxide at a rate of 0.0002 nmol/kg/hour to 2.0 nmol/kg/hour.

33. A method of producing analgesia in a subject according to claim 32, wherein the nitric oxide donor is formulated in a sustained release formulation.

34. A method of producing analgesia in a subject according to claim 32, wherein the nitric oxide donor is a slow-release nitric oxide donor.

35. A method of producing analgesia in a subject according to claim 34, wherein the effective amount of nitric oxide donor is in the range of 0.004 nmol/kg to 0.4 nmol/kg.

36. A method of producing analgesia in a subject according to claim 32, wherein the effective amount of opioid analgesic is a sub-analgesic amount.

37. A method of producing analgesia in a subject comprising administering an effective amount of an opioid analgesic with an effective amount of a slow-release nitric oxide donor or a sustained release formulation of a nitric oxide donor.

38. A method of producing analgesia in a subject according to claim 37, wherein the nitric oxide donor releases nitric oxide in the form of NO⁺ or NO⁻.

39. A method of producing analgesia in a subject according to claim 37, wherein the nitric oxide donor enhances the endogenous production of nitrosothiols.

40. A method of producing analgesia according to claim 37, wherein the nitric oxide donor reduces the endogenous production of peroxynitrite.

41. A method of producing analgesia according to claim 37, wherein the nitric oxide donor causes more endogenous production of nitrosothiols than endogenous production of peroxynitrite.

42. A method according to one of claim 32 or 37, wherein the opioid analgesic is selected from morphine, methadone, fentanyl, sufentanil, alfentanil, hydromorphone, oxymorphone, oxycodone, codeine, hydrocodeine, hydrocodone, levorphanol, meperidine, heroin, morphine-6-glucuronide, levallorphan, 6-monoacetylmorphine and tramadol.

43. A method according to one of claim 34 or 37, wherein the slow-release nitric oxide donor comprises a nitrato group coupled to a carrier compound by a linker.

44. A method according to one of claim 34 or 37, wherein the slow-release nitric oxide donor is a compound of formula (I): 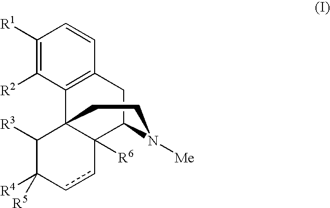
wherein R¹ is selected from OH, OC₁₋₆ alkyl, —O-A-X—NO₂, 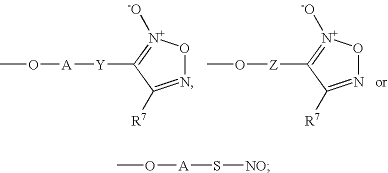
R² and R³ are each H or taken together are —O—;
R⁴ is H, OH, OC₁₋₆ alkyl, —O-A-X—NO₂, 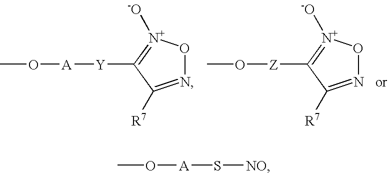
and R⁵ is H or R⁴ and R⁵ taken together form an oxo group;
R⁶ is selected from H, OH, OC₁₋₆ alkyl, —O-A-X—NO₂, 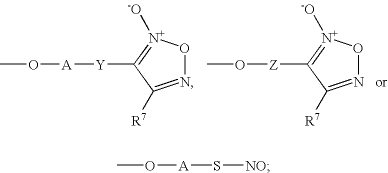 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 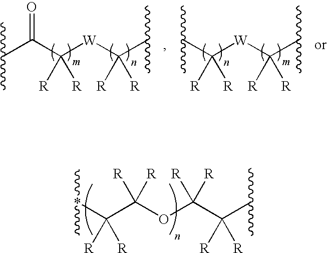
wherein W is absent or is selected from —O—, —S—, —NH—, —NC₁-6 alkyl, 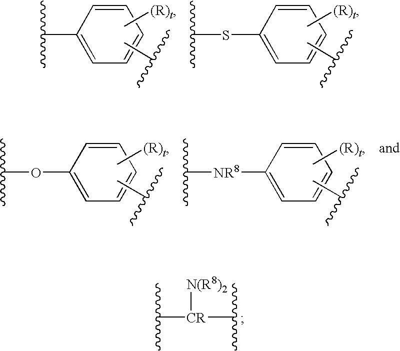
R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁-20 alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃,
B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
m is 0 or an integer from 1 to 10;
n is an integer from 1 to 10; and
t is 0 or an integer from 1 to 4.
wherein at least one of R¹, R⁴ and R⁶ is —O-A-X—NO₂, 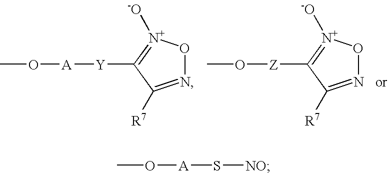
or a pharmaceutically acceptable salt thereof.

45. A method according to claim 44, wherein the slow-release nitric oxide donor is a compound of formula (II): 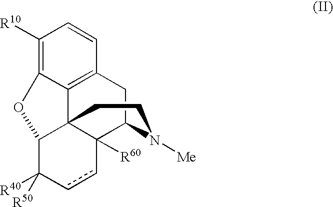
wherein R¹⁰ is selected from OH, OCH₃, —O-A-X—NO₂, 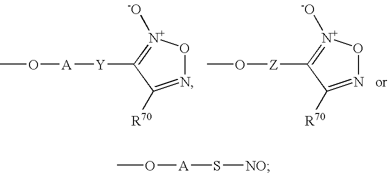
R⁴⁰ is selected from —O-A-X—NO₂, 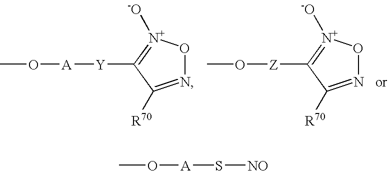
and R⁵⁰ is H or R⁴⁰ and R⁵⁰ taken together form an oxo group;
R⁶⁰ is selected from H or —O-A-X—NO₂, 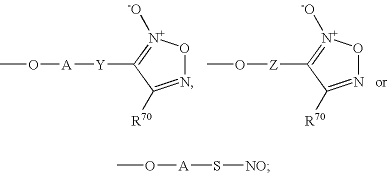 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 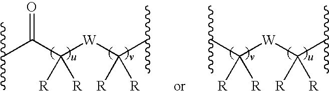
wherein W is absent or is selected from —O—, —S—, —NH—, 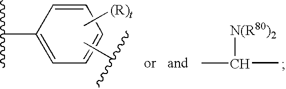
R⁷⁰ is selected from C₁— alkyl, C₁₋₆ alkoxy, C₁₋₆ alkylCO, C₁₋₆ alkylSO, C₁₋₆ alkylSO₂, phenyl, phenoxy, phenylSO, phenylSO₂, phenylCO, N(R⁸⁰)₂ and (R⁸″)₂NCO;
each R⁸⁰ is independently selected from H, C₁₋₆ alkyl, C₂₋₆ alkenyl, C₂₋₆ alkynyl or aryl;
each R is independently selected from H, C₁-6 alkyl, C₂₋₆ alkenyl, C₂₋₆ alkynyl, aryl, heterocyclyl, halo, hydroxy, C₁₋₆ alkoxy, aryloxy, C₂₋₆ alkenyloxy, heterocyclyloxy, thiol, C₁₋₆ alkylthiol, C₂₋₆ alkenylthiol, arylthiol, heterocyclylthiol, benzyl, benzyloxy, benzylthio, acyl, acyloxy, CO₂H, CO₂C₁₋₆ alkyl, SOC₁₋₆ alkyl, SO₂C₁₋₆ alkyl, SO₃H, SO₃C₁₋₆ alkyl, SONH₂, SONHC₁₋₆ alkyl, SON(C₁₋₆ alkyl)₂, SO₂NH₂, SO₂NHC₁₋₆ alkyl, SO₂N(C₁₋₆ alkyl)₂, CONH₂, CONHC₁₋₆ alkyl, CON(C₁₋₆ alkyl)₂, NH₂, NHC₁₋₆ alkyl, N(C₁₋₆ alkyl)₂, CN, CF₃ or NO₂;
u is 0 or an integer from 1 to 5;
v is an integer from 1 to 5; and
t is 0 or an integer from 1 to 4;
wherein at least one of R¹⁰, R⁴⁰ and R⁶⁰ is —O-A-X—NO₂, 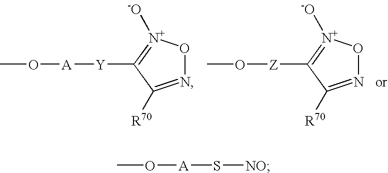
or a pharmaceutically acceptable salt thereof.

46. A method of producing analgesia according to one of claim 34 or 37, wherein the slow-release nitric oxide donor is selected from the group consisting of: 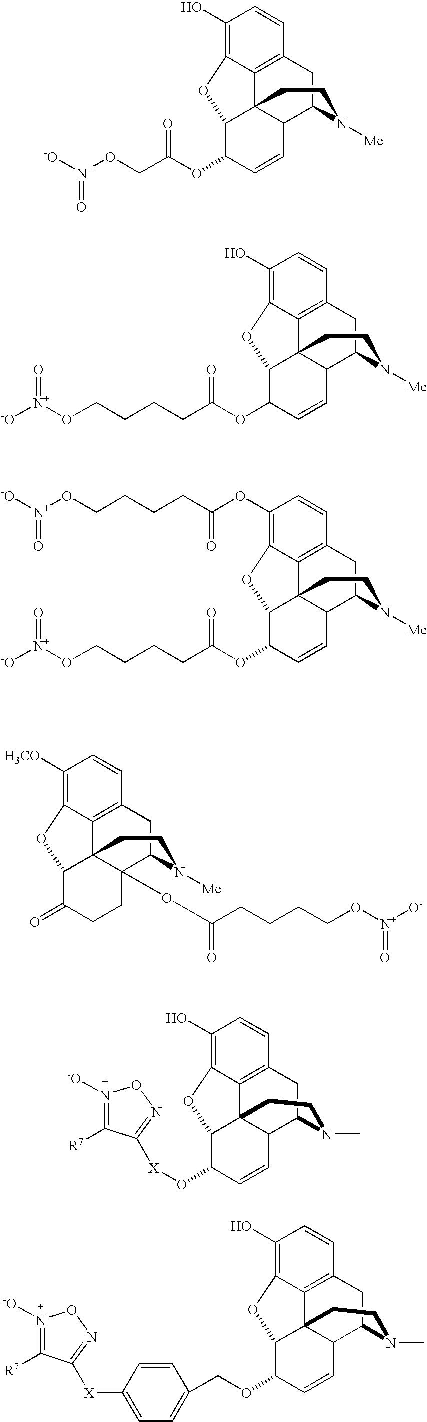 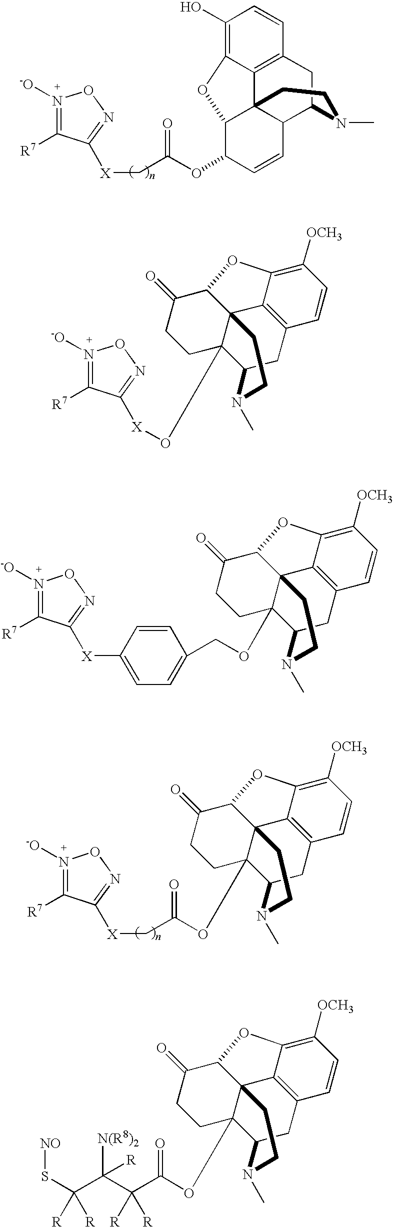 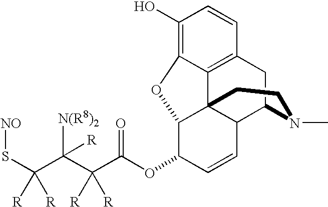
wherein R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
X is O or S; and
n is an integer from 1 to 10.

47. A method of relieving pain, comprising administering an effective amount of a nitric oxide donor and an effective amount of an opioid analgesic wherein the nitric oxide donor is a slow-release nitric oxide donor or is formulated in a sustained release formulation which delivers nitric oxide at a rate of 0.0002 nmol/kg/hour to 2.0 nmol/kg/hour.

48. A method of relieving pain comprising administering an effective amount of an opioid analgesic with an effective amount of a slow-release nitric oxide donor or a sustained release formulation of a nitric oxide donor.

49. A method according to claim 47 or 48, wherein the opioid analgesic is selected from morphine, methadone, fentanyl, sufentanil, alfentanil, hydromorphone, oxymorphone, oxycodone, codeine, hydrocodeine, hydrocodone, levorphanol, meperidine, heroin, morphine-6-glucuronide, levallorphan, 6-monoacetylmorphine and tramadol.

50. A method according to claim 47 or 48, wherein the slow-release nitric oxide donor comprises a nitrato group coupled to a carrier compound by a linker.

51. A method according to claim 47 or 48, wherein the slow-release nitric oxide donor is a compound of formula (I): 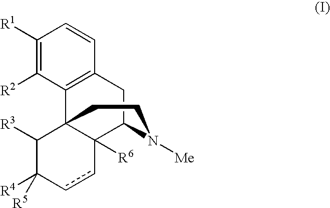
wherein R¹ is selected from OH, OC₁— alkyl, —O-A-X—NO₂, 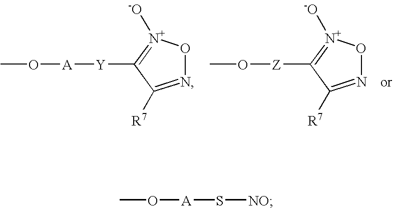
R² and R³ are each H or taken together are —O—;
R⁴ is H, OH, OC₁₋₆ alkyl, —O-A-X—NO₂, 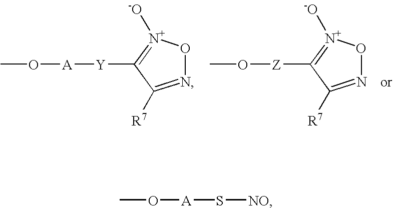
and R⁵ is H or R⁴ and R⁵ taken together form an oxo group;
R⁶ is selected from H, OH, OC₁₋₆ alkyl, —O-A-X—NO₂, 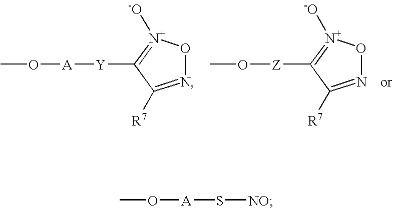 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 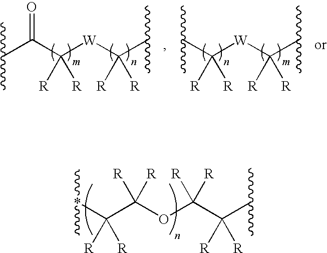
wherein W is absent or is selected from —O—, —S—, —NH—, —NC₁— alkyl 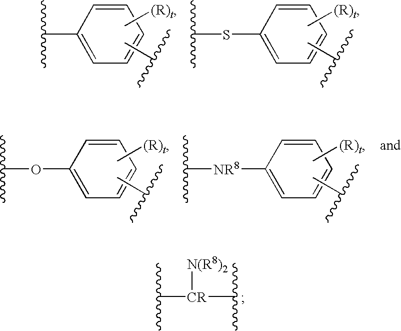
R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
m is 0 or an integer from 1 to 10;
n is an integer from 1 to 10; and
t is 0 or an integer from 1 to 4.
wherein at least one of R¹, R⁴ and R⁶ is —O-A-X—NO₂, 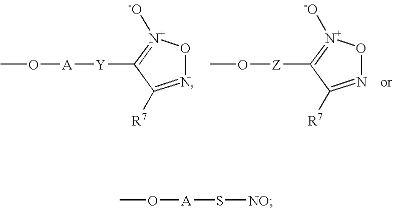
or a pharmaceutically acceptable salt thereof.

52. A method according to claim 51, wherein the slow-release nitric oxide donor is a compound of formula (II): 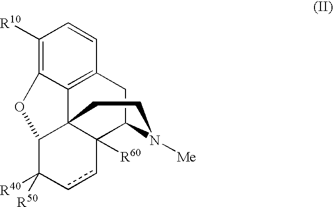
wherein R¹⁰ is selected from OH, OCH₃, —O-A-X—NO₂, 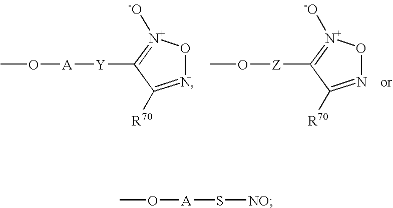
R⁴⁰ is selected from —O-A-X—NO₂, 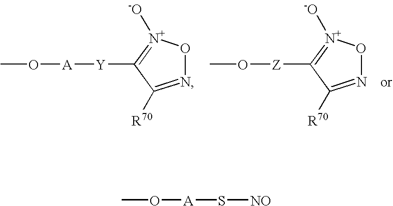
and R⁵⁰ is H or R⁴⁰ and R⁵⁰ taken together form an oxo group;
R⁶⁰ is selected from H or —O-A-X—NO₂, 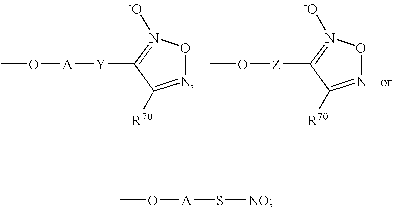 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 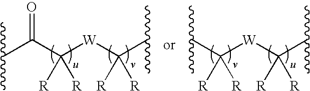
wherein W is absent or is selected from —O—, —S—, —NH—, 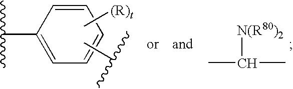
R⁷⁰ is selected from C₁₋₆ alkyl, C₁₋₆ alkoxy, C₁₋₆ alkylCO, C₁₋₆ alkylSO, C₁₋₆ alkylSO₂, phenyl, phenoxy, phenylSO, phenylSO₂, phenylCO, N(R⁸⁰)₂ and (R⁸⁰)₂NCO;
each R⁸⁰ is independently selected from H, C₁₋₆ alkyl, C₂₋₆ alkenyl, C₂₋₆ alkynyl or aryl;
each R is independently selected from H, C₁₋₆ alkyl, C₂₋₆ alkenyl, C₂₋₆ alkynyl, aryl, heterocyclyl, halo, hydroxy, C₁₋₆ alkoxy, aryloxy, C₂₋₆ alkenyloxy, heterocyclyloxy, thiol, C₁-6 alkylthiol, C₂₋₆ alkenylthiol, arylthiol, heterocyclylthiol, benzyl, benzyloxy, benzylthio, acyl, acyloxy, CO₂H, CO₂C₁₋₆ alkyl, SOC₁₋₆ alkyl, SO₂C₁₋₆ alkyl, SO₃H, SO₃C₁₋₆ alkyl, SONH₂, SONHC₁₋₆ alkyl, SON(C₁₋₆ alkyl)₂, SO₂NH₂, SO₂NHC₁₋₆ alkyl, SO₂N(C₁₋₆ alkyl)₂, CONH₂, CONHC₁₋₆ alkyl, CON(C₁₋₆ alkyl)₂, NH₂, NHC₁₋₆ alkyl, N(C₁₋₆ alkyl)₂, CN, CF₃ or NO₂;
u is 0 or an integer from 1 to 5;
v is an integer from 1 to 5; and
t is 0 or an integer from 1 to 4;
wherein at least one of R¹⁰, R⁴⁰ and R⁶⁰ is —O-A-X—NO₂, 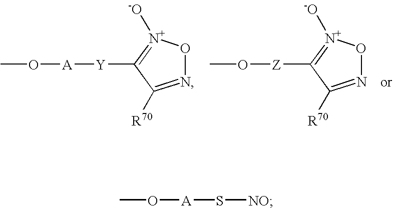
or a pharmaceutically acceptable salt thereof.

53. A method of relieving pain according to claim 47 or 48, wherein the slow-release nitric oxide donor is selected from the group consisting of: 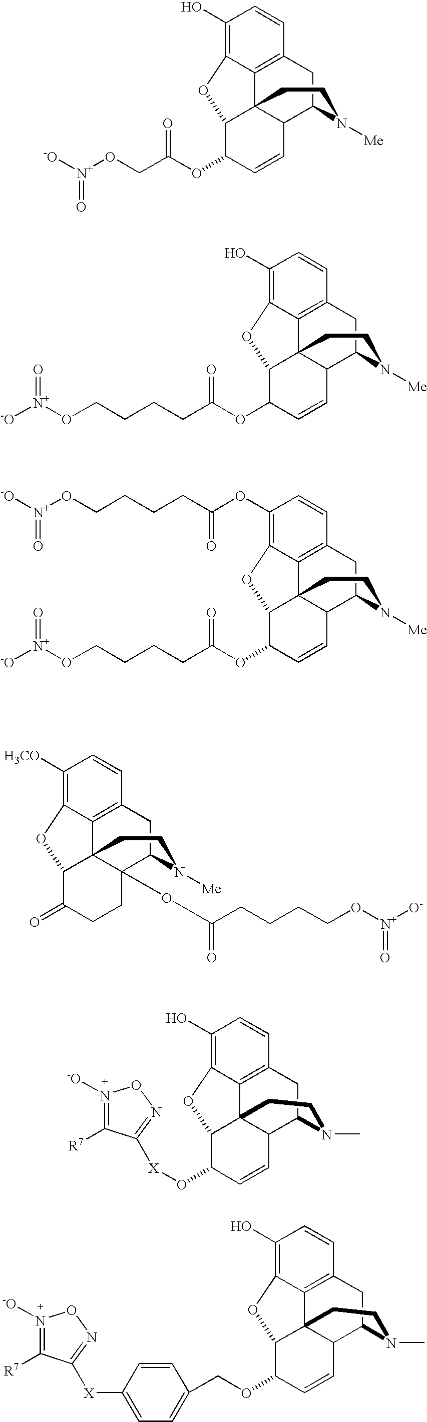 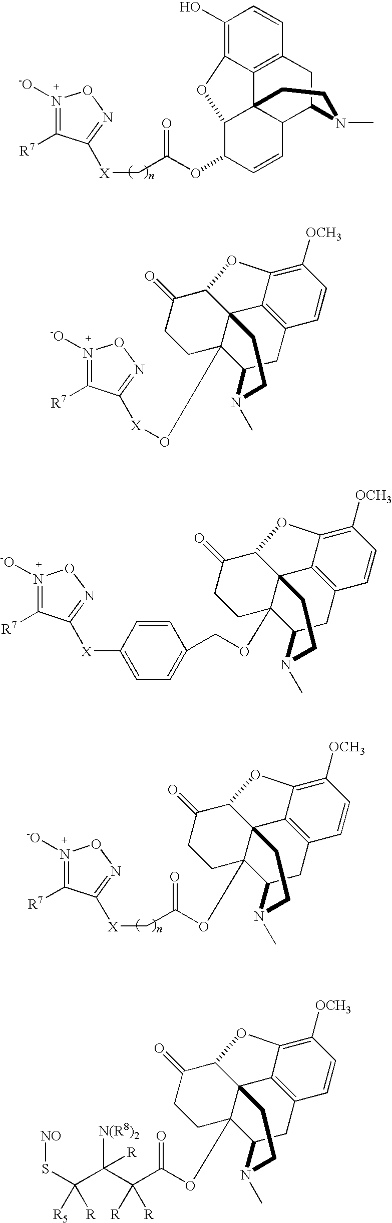 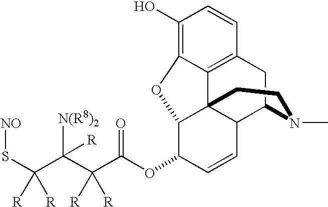
wherein R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C—20 alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
X is O or S; and
n is an integer from 1 to 10.

54. The method according to claim 47 or 48, wherein the pain is selected from the group consisting of moderate to severe cancer pain, moderate to severe post-surgical pain, pain following physical trauma, pain associated with cardiac infarction and inflammatory pain.

55. A compound of formula (I): 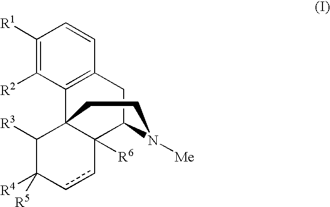
wherein R¹ is selected from OH, OC₁₋₆ alkyl, —O-A-X—NO₂, 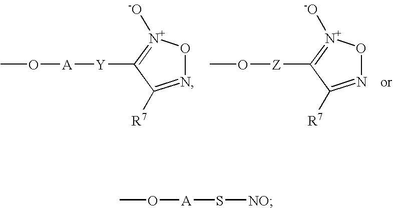
R² and R³ are each H or taken together are —O—;
R⁴ is H, OH, OC₁₋₆ alkyl, —O-A-X—NO₂, 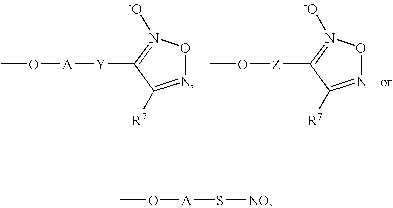
and R⁵ is H or R⁴ and R⁵ taken together form an oxo group;
R⁶ is selected from H, OH, OC₁₋₆ alkyl, —O-A-X—NO₂, 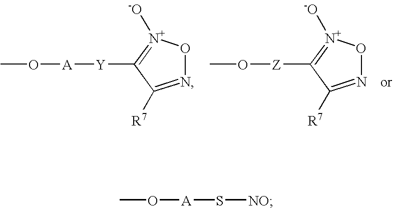 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 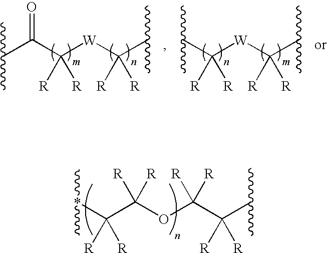
wherein W is absent or is selected from —O—, —S—, —NH—, —NC₁₋₆ alkyl, 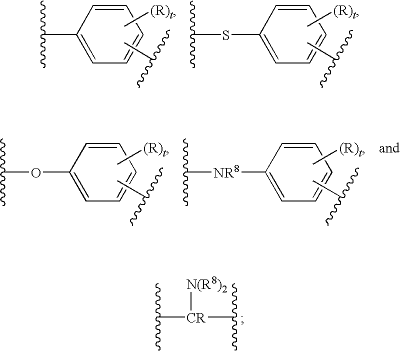
R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
m is 0 or an integer from 1 to 10;
n is an integer from 1 to 10; and
t is 0 or an integer from 1 to 4.
wherein at least one of R¹, R⁴ and R⁶ is 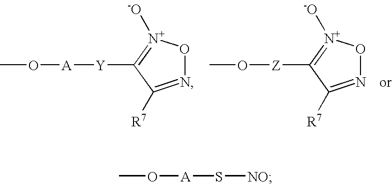
or a pharmaceutically acceptable salt thereof.

56. A compound according to claim 55, the compound being a compound of formula (II): 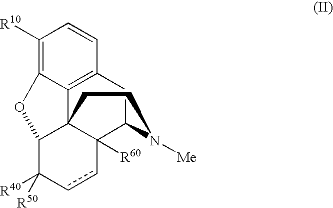
wherein R¹⁰ is selected from OH, OCH₃, —O-A-X—NO₂, 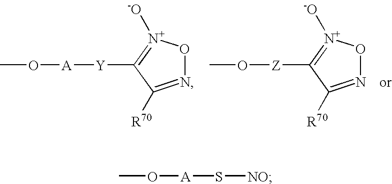
R⁴⁰ is selected from —O-A-X—NO₂, 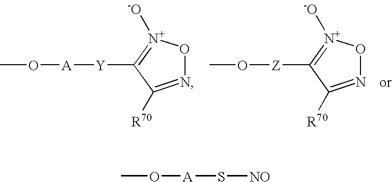
and R⁵⁰ is H or R⁴⁰ and R⁵⁰ taken together form an oxo group;
R⁶⁰ is selected from H or —O-A-X—NO₂, 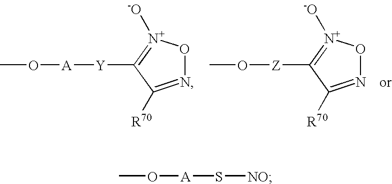 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 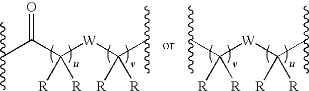
wherein W is absent or is selected from —O—, —S—, —NH—, 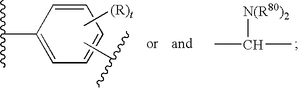
R⁷⁰ is selected from C₁₋₆ alkyl, C₁₋₆ alkoxy, C₁₋₆ alkylCO, C₁₋₆ alkylSO, C₁₋₆ alkylSO₂, phenyl, phenoxy, phenylSO, phenylSO₂, phenylCO, N(R⁸⁰)₂ and (R⁸⁰)₂NCO;
each R⁸⁰ is independently selected from H, C₁₋₆ alkyl, C₂₋₆ alkenyl, C₂₋₆ alkynyl or aryl;
each R is independently selected from H, C₁₋₆ alkyl, C₂₋₆ alkenyl, C₂₋₆ alkynyl, aryl, heterocyclyl, halo, hydroxy, C₁₋₆ alkoxy, aryloxy, C₂₋₆ alkenyloxy, heterocyclyloxy, thiol, C₁-6 alkylthiol, C₂₋₆ alkenylthiol, arylthiol, heterocyclylthiol, benzyl, benzyloxy, benzylthio, acyl, acyloxy, CO₂H, CO₂C₁₋₆ alkyl, SOC₁₋₆ alkyl, SO₂C₁₋₆ alkyl, SO₃H, SO₃C₁₋₆ alkyl, SONH₂, SONHC₁₋₆ alkyl, SON(C₁₋₆ alkyl)₂, SO₂NH₂, SO₂NHC₁₋₆ alkyl, SO₂N(C₁₋₆ alkyl)₂, CONH₂, CONHC₁₋₆ alkyl, CON(C₁₋₆ alkyl)₂, NH₂, NHC₁₋₆ alkyl, N(C₁₋₆ alkyl)₂, CN, CF₃ or NO₂;
u is 0 or an integer from 1 to 5;
v is an integer from 1 to 5; and
t is 0 or an integer from 1 to 4;
wherein at least one of R¹⁰, R⁴⁰ and R⁶⁰ is 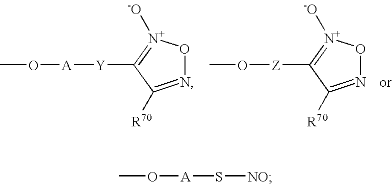
or a pharmaceutically acceptable salt thereof.

57. A compound selected from the group consisting of: 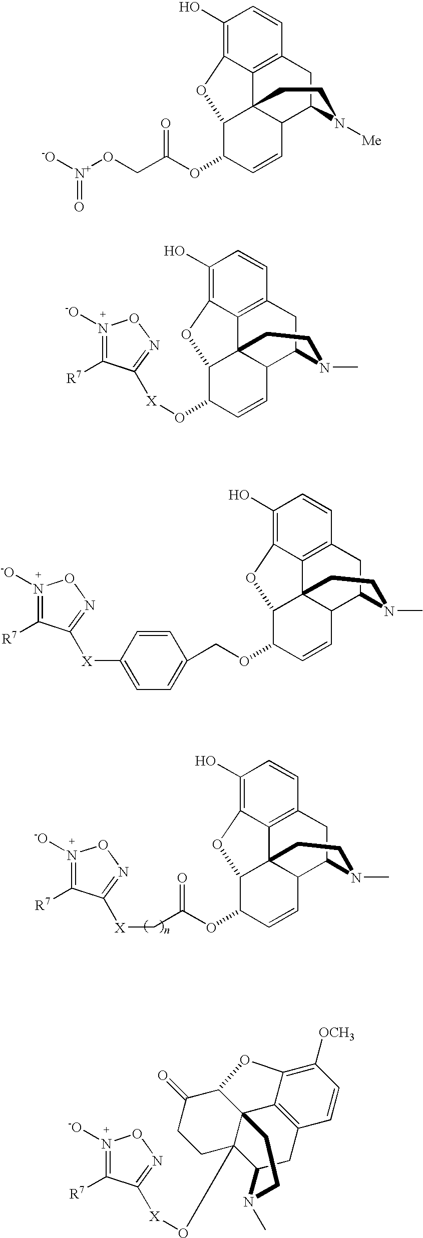 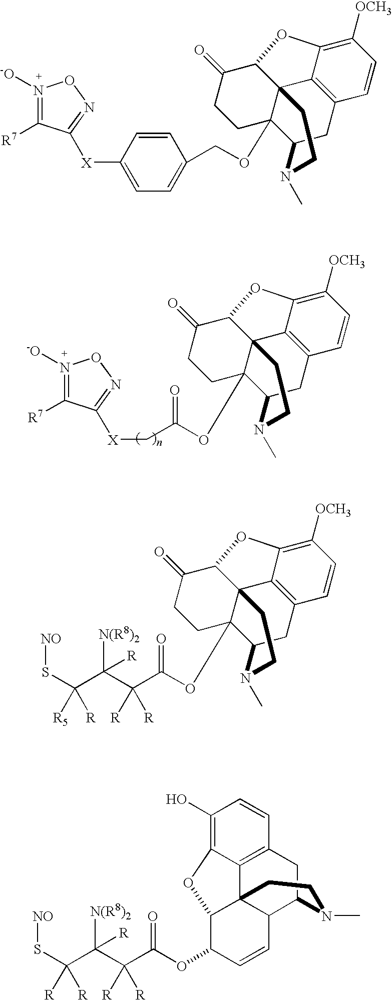
wherein R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁-20 alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
X is O or S; and
n is an integer of 1 to 10.

58. A pharmaceutical composition comprising a compound of formula (I): 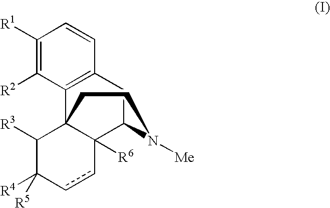
wherein R¹ is selected from OH, OC₁₋₆ alkyl, —O-A-X—NO₂, 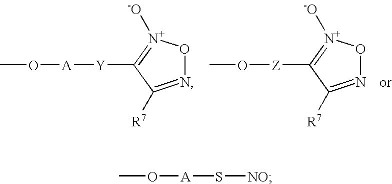
R² and R³ are each H or taken together are —O—;
R⁴ is H, OH, OC₁₋₆ alkyl, —O-A-X—NO₂, 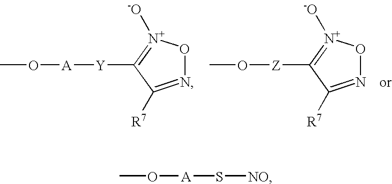
and R⁵ is H or R⁴ and R⁵ taken together form an oxo group;
R⁶ is selected from H, OH, OC₁₋₆ alkyl, —O-A-X—NO₂, 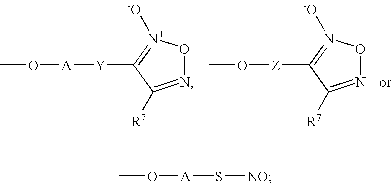 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 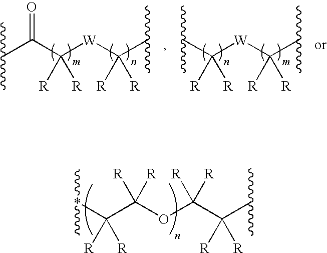
wherein W is absent or is selected from —O—, —S—, —NH—, —NC₁— alkyl, 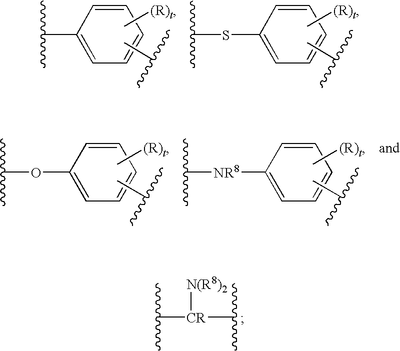
R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
m is 0 or an integer from 1 to 10;
n is an integer from 1 to 10; and
t is 0 or an integer from 1 to 4.
wherein at least one of R¹, R⁴ and R⁶ is 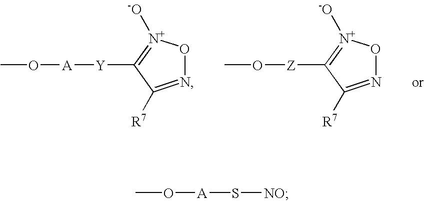
or a pharmaceutically acceptable salt thereof,
and a pharmaceutically acceptable carrier, excipient or diluent.

59. A pharmaceutical composition according to claim 58 comprising a compound of formula (II): 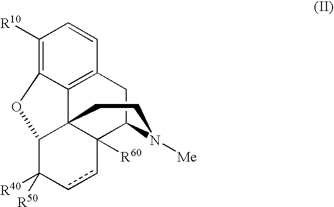
wherein R¹⁰ is selected from OH, OCH₃, —O-A-X—NO₂, 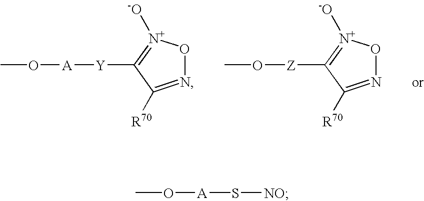
R⁴⁰ is selected from —O-A-X—NO₂, 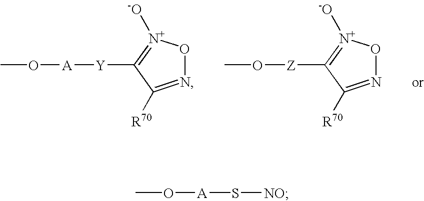
and R⁵⁰ is H or R⁴⁰ and R⁵⁰ taken together form an oxo group;
R⁶⁰ is selected from H or -A-X—NO₂, 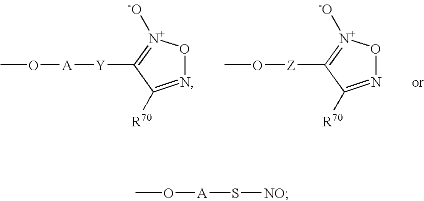 [symbol]
represents a single or double bond;
X represents O or S;
Y represents O, S, SO, SO₂, CO, CONH, CO₂, NH or NC₁₋₆ alkyl;
Z represents SO, SO₂, CO, CONH, CO₂, NH or NC₁₋₆ alkyl;
A represents 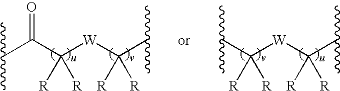
wherein W is absent or is selected from —O—, —S—, —NH—, 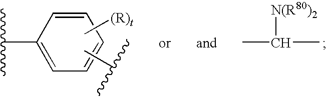
R⁷⁰ is selected from C₁₋₆ alkyl, C₁₋₆ alkoxy, C₁₋₆ alkylCO, C₁₋₆ alkylSO, C₁₋₆ alkylSO₂, phenyl, phenoxy, phenylSO, phenylSO₂, phenylCO, N(R⁸⁰)₂ and (R⁸⁰)₂NCO;
each R⁸⁰ is independently selected from H, C₁₋₆ alkyl, C₂₋₆ alkenyl, C₂₋₆ alkynyl or aryl;
each R is independently selected from H, C₁₋₆ alkyl, C₂₋₆ alkenyl, C₂₋₆ alkynyl, aryl, heterocyclyl, halo, hydroxy, C₁₋₆ alkoxy, aryloxy, C₂₋₆ alkenyloxy, heterocyclyloxy, thiol, C₁₋₆ alkylthiol, C₂₋₆ alkenylthiol, arylthiol, heterocyclylthiol, benzyl, benzyloxy, benzylthio, acyl, acyloxy, CO₂H, CO₂C₁₋₆ alkyl, SOC₁₋₆ alkyl, SO₂C₁₋₆ alkyl, SO₃H, SO₃C₁₋₆ alkyl, SONH₂, SONHC₁₋₆ alkyl, SON(C₁₋₆ alkyl)₂, SO₂NH₂, SO₂NHC₁₋₆ alkyl, SO₂N(C₁₋₆ alkyl)₂, CONH₂, CONHC₁₋₆ alkyl, CON(C₁₋₆ alkyl)₂, NH₂, NHC₁₋₆ alkyl, N(C₁₋₆ alkyl)₂, CN, CF₃ or NO₂;
u is 0 or an integer from 1 to 5;
v is an integer from 1 to 5; and
t is 0 or an integer from 1 to 4;
wherein at least one of R¹⁰, R⁴⁰ and R⁶⁰ is 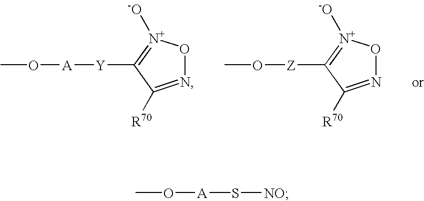
or a pharmaceutically acceptable salt thereof.

60. A pharmaceutical composition comprising a compound selected from the group consisting of: 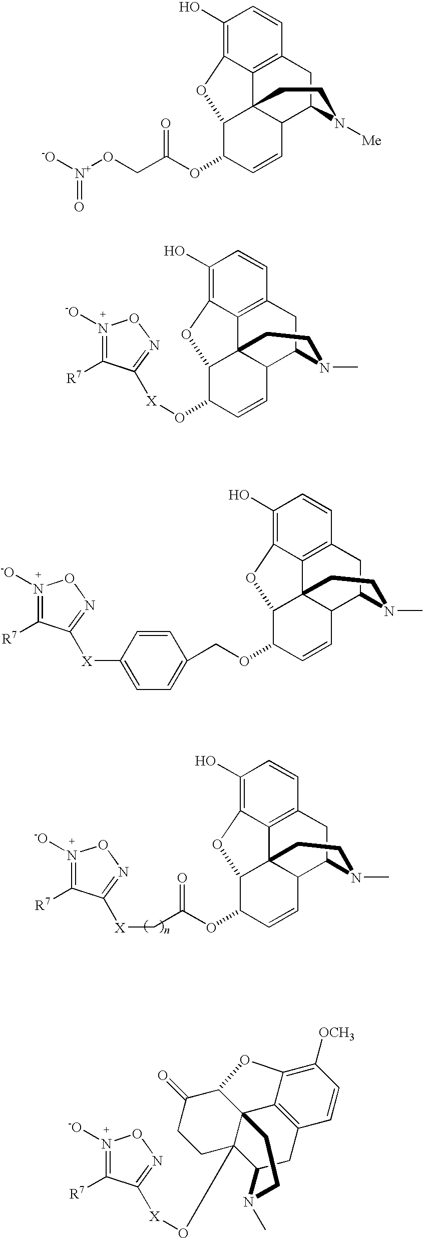 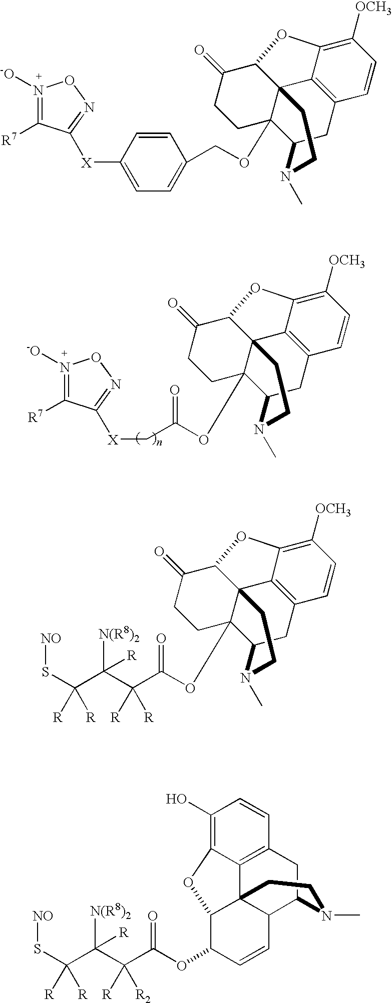
wherein R⁷ is selected from C₁₋₂₀ alkyl, C₁₋₂₀ alkoxy, C₁₋₂₀ alkylCO, C₁₋₂₀ alkylSO, C₁₋₂₀ alkylSO₂, aryl, aryloxy, arylSO₂, arylSO, arylCO, N(R⁸)₂, (R⁸)₂NCO;
each R⁸ is independently selected from H, C₁₋₂₀ alkyl, C₂₋₂₀ alkenyl, C₂₋₂₀ alkynyl or aryl;
each R is independently selected from H, C₁₋₂₀ alkyl, C₂₋₂₀ alkenyl, C₂₋₂₀ alkynyl, aryl, heterocyclyl, halo, hydroxy, C₁₋₂₀ alkoxy, aryloxy, C₂₋₂₀ alkenyloxy, C₂₋₂₀ alkynyloxy, heterocyclyloxy, thiol, C₁₋₂₀ alkylthiol, C₂₋₂₀ alkenylthiol, C₂₋₂₀ alkynylthiol, arylthiol, heterocyclylthiol, benzyl, benzyloxy, benzylthio, acyl, acyloxy, CO₂R′, SOR′, SO₂R′, SO₃R′, SON(R′)₂, SO₂N(R′)₂, SO₃N(R′)₂, CON(R′)₂, N(R′)₂, P(R′)₃, P(═O)(R′)₃, Si(R′)₃, B(R′)₂C₁₋₂₀ alkyl, CN, CF₃ or NO₂ where each R′ is independently selected from H, C₁₋₂₀ alkyl, C₂₋₂₀ alkenyl, C₂₋₂₀ alkynyl, aryl and heterocyclyl;
X is O or S; and
n is an integer from 1 to 10.

61. A pharmaceutical composition according to claim 58 or 60 further comprising an opioid analgesic.

62. A pharmaceutical composition according to claim 61, wherein the opioid analgesic is selected from morphine, methadone, fentanyl, sufentanil, alfentanil, hydromorphone, oxymorphone, oxycodone, codeine, hydrocodeine, hydrocodone, levorphanol, meperidine, heroin, morphine-6-glucuronide, levallorphan, 6-monoacetylmorphine and tramadol.
